(19) United States
(12) Patent Application Publication
(10) Pub. No.: US 2022/0031220 A1
(43) Pub. Date: Feb. 3, 2022

(54) MODEL-BASED SENSOR TECHNOLOGY FOR DETECTION OF CARDIOVASCULAR STATUS

(71) Applicant: THE CURATORS OF THE UNIVERSITY OF MISSOURI, Columbia, MO (US)

(72) Inventor: Giovanna GUIDOBONI, Chesterfield, MO (US)

(21) Appl. No.: 17/278,835

(22) PCT Filed: Sep. 24, 2019
(86) PCT No.: PCT/US19/52738

§ 371 Date: Mar. 23, 2021

Related U.S. Application Data
(60) Provisional application No. 62/735,716, filed on Sep. 24, 2018.

(51) Int. Cl. A61B 5/319; A61B 5/0205; A61B 5/021; A61B 5/024; A61B 5/026; A61B 5/11; A61B 5/352; A61B 5/36

(52) U.S. Cl. CPC A61B 5/319; A61B 5/0205; A61B 5/021; A61B 5/024; A61B 2560/02; A61B 5/1102; A61B 5/352; A61B 5/36; A61B 2562/0219; A61B 5/026

(57) ABSTRACT

The invention disclosed here is sensor technology incorporating a closed-loop mathematical model of a cardiovascular system, for detection and/or monitoring of a cardiovascular status of a test subject. The closed-loop cardiovascular model equates common circuit components with real-world cardiovascular parameters, and is capable of tracking and/or predicting cardiovascular behavior.

### CROSS-REFERENCE AND PRIORITY CLAIM TO RELATED APPLICATIONS

This application claims priority to U.S. provisional patent application No. 62/735,716, filed Sep. 24, 2018, which is incorporated herein by reference in its entirety.

### BACKGROUND

In recent years, there has been a resurgence of research into physiological signals that are representative of certain cardiovascular functions, as new sensing devices allow for easier, non-invasive capture of such signals. Likewise there has been renewed interest in techniques for obtaining, analyzing and processing such signals. Such techniques and signals include, but are not limited to, electrocardiography (e.g., an electrocardiogram (ECG)), ballistocardiography (e.g., a ballistocardiogram (BCG)), and seismocardiography (e.g., a seismocardiogram (SCG)). For example, electrocardiography produces an ECG of the electrical activity of the heart using electrodes placed on the skin. The ECG may take the form of a graph of voltage versus time. These electrodes detect electrical changes that result from cardiac muscle depolarization (e.g., contraction/electrical activity) followed by repolarization (e.g., rest/no electrical activity) during each cardiac cycle (e.g., heartbeat). Abnormal ECG patterns occur in various cardiac abnormalities, such as cardiac rhythm disturbances (e.g., atrial fibrillation and ventricular tachycardia), inadequate coronary artery blood flow (e.g., myocardial ischemia and myocardial infarction), and electrolyte disturbances (e.g., hypokalemia and hyperkalemia). There are three main components to an ECG: the P wave, which represents the depolarization of the (right and left) atria; the QRS complex, which represents the depolarization of the (right and left) ventricles; and the T wave, which represents the repolarization of the ventricles. By measuring time intervals on the ECG, it can be determined how long the electrical wave takes to pass through the heart, an indication of if the electrical activity of the heart is normal, slow, fast or irregular. Additionally, by measuring the amount of electrical activity passing through the heart muscle, a cardiologist may be able to find out if parts of the heart are too large or are overworked.

Ballistocardiography captures the signal generated by the repetitive motion of the human body due to sudden ejection of blood into the great vessels with each heartbeat. A BCG signal is generated as a result of the dynamics of blood flow through the cardiovascular system. While ECG has been the more common cardiovascular monitoring technique (e.g., due in part to the difficulty involved in detecting and analyzing BCG waveforms), there has been renewed interest in BCG in recent years, sparked by the increase in computing power and signal processing techniques. Compared to ECG techniques which require a plurality of electrodes/leads to be attached to the patient for testing and detection, BCG techniques are well-suited for non-invasive (e.g., passive) continuous patient monitoring. For example, there exist bed, chair, and other sensors to capture the BCG. While much of this work has focused on monitoring heart rate along with respiration rate from the accompanying respiration signal and other parameters for tracking sleep quality, recent work has also investigated the BCG waveform morphology for the purpose of tracking changes in cardiovascular health. This cardiovascular application offers a special relevance and significant potential in monitoring older adults as they age. Identifying very early signs of cardiovascular health changes provides an opportunity for very early treatments before health problems escalate, where very early treatment offers better health outcomes and the potential to avoid hospitalizations.

However, one challenge in using the BCG waveform to track cardiovascular health changes is the lack of a standardized measurement device and protocol and the lack of uniform clinical interpretation. Theoretical foundations exist for interpreting BCG signals by expressing the displacement of the center of mass of the human body as a function of the blood volumes occupying different vascular compartments at a given time during the cardiac cycle. A coordinate Γ of the center of mass of the body along the head-to-toe direction at any given time t can be written as Equation (1) below:

$$Γ ( t ) = \frac{{ρ}_{b}}{M} {\sum }_{i = 1}^{N} {V}_{i} ( t ) {y}_{i} + c \qquad ( 1 )$$

In Equation (1), ρ_{b} is the blood density, M is the body mass, N is the total number of vascular compartments considered in the model and c is a constant term representing the body frame. Each vascular compartment i, with i=1, . . . , N, is assumed to be located at the fixed coordinate yᵢ and to be filled with the blood volume Vᵢ(t) at time t. Since the term c in Equation (1) is constant for a given person, the BCG signal associated with the center of mass displacement in the head-to-toe direction is defined as shown in Equation (2):

$${BCG}_{disp} ( t ) \text{:=} \frac{{ρ}_{b}}{M} {\sum }_{i = 1}^{N} {V}_{i} ( t ) {y}_{i} \qquad ( 2 )$$

The BCG signals associated with velocity and acceleration of the center of mass can be obtained via time-differentiation as shown in Equations (3) and (4), respectively:

$$\begin{matrix}{BCG}_{vel} ( t ) \text{:=} \frac{{ρ}_{b}}{M} {\sum }_{i = 1}^{N} \frac{{dV}_{i} ( t )}{dt} {y}_{i} & ( 3 ) \\ {BCG}_{acc} ( t ) \text{:=} \frac{{ρ}_{b}}{M} {\sum }_{i = 1}^{N} \frac{{d}^{2} {V}_{i} ( t )}{{dt}^{2}} y & ( 4 )\end{matrix}$$

In the human body, the waveforms Vᵢ(t) result from the complex interplay between the blood volume ejected from the heart, the resistance to flow that blood experiences across the cardiovascular system and the pressure distribution within it. Prior techniques have characterized the volume waveforms Vᵢ(t) by means of experimental measurements at each location yᵢ. While there were some early computer-aided approaches for quantitative interpretation of BCG signals (e.g., where the electric analogy to fluid flow was leveraged to describe the motion of blood through the arterial system during the cardiac cycle and calculate the resulting BCG signal), there has since been little interest in the topic of theoretical interpretation of BCG signals, until recently.

Another BCG approach utilized a three-dimensional finite element model for blood flow in the thoracic aorta to show that the traction at the vessel wall appears of similar magnitude to recorded BCG forces. Yet another approach proposed a simplified model based on the equilibrium forces within the aorta to show that blood pressure gradients in the ascending and descending aorta are major contributors to the BCG signal.

Despite these different blood flow models, the aforementioned approaches share the common feature of focusing only on the arterial side of the cardiovascular system, thereby leading to open-loop models of the circulation. In reality, human blood circulates within a closed-loop system and, as a consequence, hemodynamic changes observed at the level of the major arteries might be a result of changes occurring elsewhere within the closed-loop system. For example, left ventricular heart failure leads to an increase in fluid pressure that is transferred back to the lungs, ultimately damaging the right side of the heart and causing right heart failure. Another example is given by venothromboembolism, a disorder manifested by deep vein thrombosis and pulmonary embolism. Deep vein thrombosis occurs when a blood clot forms in a vein, most often in the leg, but they can also form in the deep veins of the arm, splanchnic veins, and cerebral veins. A pulmonary embolism occurs when a clot breaks loose and travels to the pulmonary circulation, causing thrombotic outflow obstruction and a sudden strain on the right ventricle. Sequelae of such an event include decreased right ventricular cardiac output, poor overall cardiac output, and decreased systemic blood pressure. Thus, current techniques for BCG analysis and relation to cardiovascular events is lacking.

Seismocardiography is the non-invasive measurement of accelerations in the chest wall produced by myocardial movement. The SCG signal is representative of ventricular contractions which trigger vibrations of the heart walls, valves, and blood, and which propagate to the surface of the chest. Similar to BCG techniques, SCG techniques are well-suited for non-invasive (e.g., passive) continuous patient monitoring. For example, SCG techniques use a sensor (e.g., accelerometer) that is small in size, low in weight, and hence conducive to wearable applications.

The various ECG, BCG and SCG signals are useful in the detection and monitoring of certain cardiovascular events that have been shown to be representative of certain cardiovascular functions and/or conditions. For example, main heart malfunctions, such as congestive heart failure and valvular disease, have been shown to alter the BCG signal. Such alterations of the BCG signal can be detected via sensors, thereby allowing for passive, non-contact monitoring of cardiovascular status. Such sensors can be positioned, for example, under a bed or on an armchair.

As another example, ventriculoarterial coupling (VAC) is known to reflect the mechanoenergetic performance of the heart and provides an important clinical biomarker for the management of several pathological conditions, including shock, hypertension and heart failure. VAC is calculated as the ratio of the effective arterial elastance (Eₐ) to the end-systolic elastance (Eₛ), namely VAC=Eₐ/Eₛ, and is currently estimated from a pressure-volume curve. However, such estimation of VAC via a pressure-volume curve suffers from several drawbacks that limits its widespread application in a clinical context. First of all, this technique requires invasive measurements of pressure and volume in the left ventricle, which may be obtained by inserting a cannula or a catheter subcutaneously. In addition, Eₐ is not a pure index of arterial load and it is insensitive to the pulsatile flow. Thus, there is a need for improvement in VAC assessment.

While current models and techniques are capable of acquiring and interpreting signals such as a BCG/ECG/SCG signal, such models include only the arterial side of blood circulation, referred to herein as open-loop models. Such known models are therefore unsuitable for simulating aspects of closed-loop, real-life blood circulation. That is, current models and techniques fail to provide for a complete mechanistic description of the relationship between signals such as BCG, ECG and SCG signals and cardiovascular mechanisms and/or functions.

Moreover, while current sensors such as accelerometers are used to detect, for example, an SCG signal, such sensors are generalized, and thus are not tuned to detect particularized cardiovascular functions or particularized physiology of an individual patient. For example, in the case of BCG, a root BCG signal that is due to body motion translates into different measured BCG signals depending on the physical properties of the measuring device (e.g., a sensor). Accordingly, conventional sensor technology is insufficient for providing nuanced detection and/or monitoring of particularized cardiovascular functions.

### SUMMARY

The inventors disclose herein examples of innovative technology that overcome such shortcomings in conventional techniques and conventional technology. For example, the inventors disclose computer systems that are programmed to provide a mechanistic description/relationship that characterizes signals such as BCG, SCG and/or ECG signals. The inventors also disclose sensors that can be tuned based on such mechanistic relationships and are thus able to be used for particularized detection and/or monitoring of cardiovascular functions.

Because human blood circulates within a closed-loop system, the above-noted conventional open-loop modelling is an insufficient methodology for modelling of real-life blood circulation and related cardiovascular functions. Due to the closed-loop system, hemodynamic changes observed at the level of the heart and the major arteries are suggestive of changes occurring elsewhere within the closed-loop system. In order to account for these important feedback mechanisms within the human circulatory system, example embodiments described herein are able to interpret signals by implementing a novel closed-loop mathematical model of the cardiovascular system.

More specifically, the closed-loop model disclosed herein models physiological cardiovascular parameters and functions. Blood circulation can be modeled using an analogy between electric systems and hydraulic networks. In this context, electric potentials correspond to fluid pressure, electric charges correspond to fluid volumes and electric currents correspond to volumetric flow rates. The closed-loop model developed by the inventors comprises a network of resistors, capacitors, inductors, voltage sources and switches arranged into four main interconnected compartments representing the heart, the systemic circulation, the pulmonary circulation and the cerebral circulation. This improved mathematical modelling of the cardiovascular system and cardiovascular functions is able to interpret relationships between cardiovascular functions, BCGs, SCGs, etc., via the mathematical modeling. The cardiovascular model is simulated via software to simulate cardiovascular mechanisms. The software can be calibrated on physiological parameters for an average human body, and/or can be calibrated based on sex-specific male and female physiological parameters. The software can further be customized to specific needs, e.g., individualized parameters for each patient.

For example, with respect to BCG, the waveforms Vᵢ(t) can be calculated according to a mathematical model based on the physical principles governing vascular physiology, thereby paving the way for the use of quantitative methods to interpret BCG signals and identify cardiovascular abnormalities in a given patient.

The resulting accurate and predictive modeling can be applied to develop improved sensor technology that can be tuned to detect a particular cardiovascular disease (CVD) and/or tuned to individualized parameters of a CVD patient. Since the progression of CVD patients is often gradual and subtle, earlier detection of disease progression is needed in order to intervene effectively to optimize the patient's quality of life and survival. The mechanistic description of the modeling described herein provides for the development of innovative indicators applied to sensor technology to monitor disease progression and/or detect disease in patients (e.g., outside of the hospital setting), thereby improving both early detection and continuous monitoring of cardiovascular disease in an unobtrusive manner. This represents a significant improvement over conventional cardiovascular modelling techniques and conventional sensor technology.

Such a novel technique according to one aspect of the present disclosure allows for model-based tuning of a sensor, thereby providing for a sensor that has tuned detection capability for detection of one or more cardiovascular functions, and providing for the sensor to be used in a passive, non-contact (e.g., non-invasive) manner during monitoring of cardiovascular status (e.g., of a patient). The model-based sensor tuning can likewise provide for tuning a sensor to detect individualized physiological parameters of a patient such as a CVD patient. Thus, the aforementioned closed-loop model of the present application, when integrated with sensor technology, provides for tuned sensors capable of accurately detecting a particular cardiovascular disorder and/or effectively monitoring (e.g., individualized) cardiovascular status, representing clear improvement and advancement over conventional techniques.

The aforementioned general and specific aspects may be implemented as a device, a method, a system, and a computer program, or any selective combination of each.

### BRIEF DESCRIPTION OF THE DRAWINGS

The accompanying drawings, which are incorporated in and form a part of the specification, illustrate the embodiments of the present invention and together with the description, serve to explain the principles of the invention. In the drawings:

FIG. 1 illustrates a closed-loop cardiovascular model according to one embodiment of the present invention;

FIG. 2 illustrates an experimental test setup for carrying out cardiovascular measurements of a test subject;

FIG. 3 illustrates a plot of a qualitative description of left ventricular function simulated via the embodiment of FIG. 1;

FIG. 4 illustrates a Wiggers diagram of ventricular function;

FIGS. 5A to 5F illustrate pressure waveforms simulated via the embodiment of FIG. 1, where FIG. 5A illustrates an ascending aorta comparison, FIG. 5B illustrates an aortic arch comparison, FIG. 5C illustrates an abdominal aorta comparison, FIG. 5D illustrates a common carotid aorta comparison, FIG. 5E illustrates a thoracic aorta comparison, and FIG. 5F illustrates a common iliac artery comparison;

FIG. 6 illustrates a waveform f_{A}(t) simulated via the embodiment of FIG. 1;

FIG. 7 illustrates ballistocardiogram waveforms for displacement, velocity and acceleration;

FIGS. 8A to 8G illustrate electrical elements according to the embodiment of FIG. 1, where FIG. 8A illustrates a linear resistor, FIG. 8B illustrates a linear resistor with ideal switch, FIG. 8C illustrates a linear capacitor, FIG. 8D illustrates a variable capacitor for arterial viscoelasticity, FIG. 8E illustrates a variable capacitor for ventricular elastance, FIG. 8F illustrates a voltage source, and FIG. 8G illustrates a linear inductor;

FIGS. 9A and 9B illustrate a sensor system used with the embodiment of FIG. 1, where FIG. 9A illustrates a sensor system comprising separate components, and FIG. 9B illustrates a sensor system with integral components;

FIG. 10 illustrates a diagram of a method of acquisition and comparison of force data using the embodiment of FIG. 1;

FIG. 11 illustrates a closed-loop cardiovascular model according to another embodiment of the present invention;

FIG. 12 illustrates a conventional pressure-volume curve characterizing the efficiency of the left ventricle;

FIG. 13 illustrates plots of an electrocardiogram signal, a synchronous seismocardiogram signal, and heart sounds generated by bandpass filtering of the seismocardiogram signal according to the embodiment of FIG. 11;

FIGS. 14A and 14B illustrate circuit representations according to the embodiment of FIG. 11, where FIG. 14A illustrates circuit representations of ventricles and valves, and FIG. 14B illustrates circuit representations of large arteries;

FIG. 15 illustrates plots of a sample ECG signal and an activation function according to the embodiment of FIG. 11;

FIG. 16 illustrates timing plots of the embodiment according to FIG. 11;

FIGS. 17A and 17B illustrate pressure-volume curves, where FIG. 17A illustrates known pressure-volume curves; and FIG. 17B illustrates pressure-volume curves simulated via the embodiment of FIG. 11;

FIGS. 18A and 18B illustrate pressure-volume curves with respect to reduced afterload, where FIG. 18A illustrates known pressure-volume curves; and FIG. 18B illustrates pressure-volume curves simulated via the embodiment of FIG. 11;

FIG. 19 illustrates a sample segment of electrocardiogram and seismocardiogram signals and a filtered heart sound acquired from the seismocardiogram in connection with the embodiment of FIG. 11;

FIG. 20 illustrates simultaneous recordings of electrocardiogram and seismocardiogram signals in connection with the embodiment of FIG. 11;

FIG. 21 illustrates a comparison between valve timings estimated with the embodiment of FIG. 11 and those obtained via conventional analysis;

FIG. 22 illustrates filtered seismocardiogram signals in connection with the embodiment of FIG. 11;

FIG. 23 illustrates predicted values of LVET, Q_{MV} and Q_{AV} in connection with the embodiment of FIG. 11;

FIG. 24 illustrates predicted values of LVET, Q_{MV} and Q_{AV} in connection with the embodiment of FIG. 11;

FIG. 25 illustrates a process flow for an embodiment of implementing the closed loop cardiovascular models to improve the monitoring and detection of cardiac status;

FIG. 26 illustrates a process flow for an embodiment of implementing the closed loop cardiovascular models to improve the monitoring and detection of a particular cardiac status; and

FIG. 27 illustrates a table of parameter values for the closed-loop model according to the embodiment of FIG. 1.

### DETAILED DESCRIPTION OF EXAMPLE EMBODIMENTS

Referring to the accompanying drawings in which like reference numbers indicate like elements, one embodiment of the present application is directed to formulation and application of a novel closed-loop physiological cardiovascular model for interpretation of a BCG signal, such model being capable of being used to tune various sensor devices for improved detection and/or monitoring of cardiovascular functions.

In order to account for the above-discussed important feedback mechanisms within the human circulatory system, the present application provides a novel quantitative interpretation of the BCG signal by means of a closed-loop mathematical model for the cardiovascular system. While other modeling approaches have been proposed in cardiovascular research, including some that have highlighted the non-negligible effect of the feedback wave within a closed-loop circuit, the inventors have developed a novel closed-loop model for the cardiovascular system that includes sufficient circuit elements to reproduce theoretically the BCG signal. The model is therefore able to (i) reproduce major features of cardiovascular physiology via comparison with published experimental and clinical data, and (ii) predict BCG signals associated with the center of mass displacement, velocity and acceleration assessed via comparison with known experimental BCG waveforms and BCG data acquired by experimentation (e.g., by way of a three-axis accelerometer placed on a suspended bed).

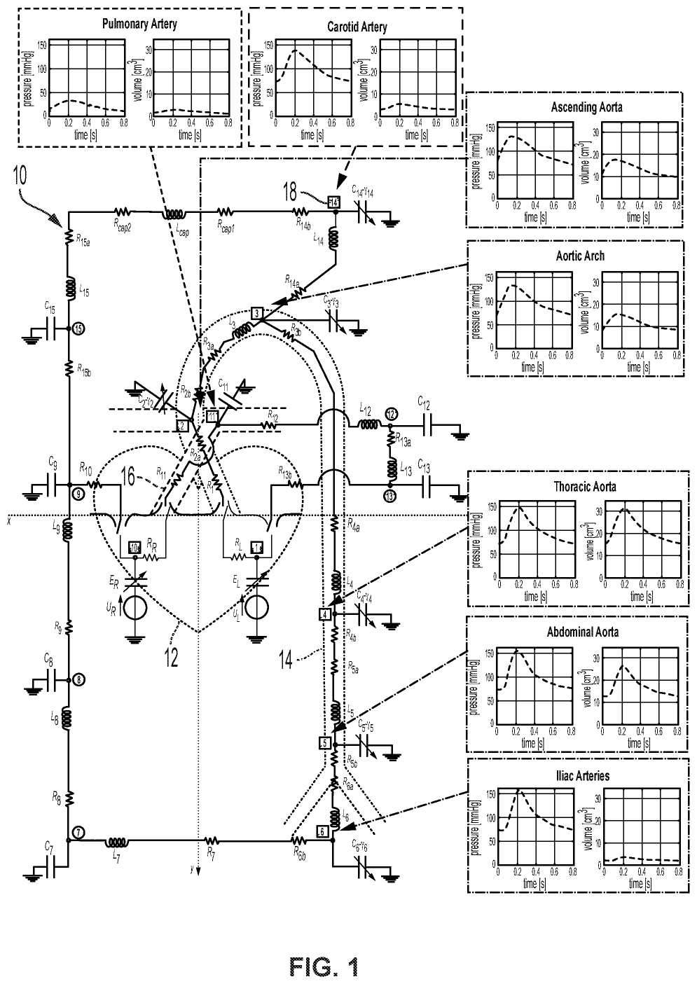

Blood circulation is modeled using the analogy between electric systems and hydraulic networks. In this context, electric potentials correspond to fluid pressure, electric charges correspond to fluid volumes and electric currents correspond to volumetric flow rates. As shown in FIG. 1, the closed-loop cardiovascular model 10 developed by the inventors includes a network of resistors, capacitors, inductors, voltage sources and switches arranged into four main interconnected compartments representing the heart, the systemic circulation, the pulmonary circulation, and the cerebral circulation. This schematic representation of the closed-loop mathematical model describing the flow of blood through the circulatory system leverages the electric analogy to fluid flow, where resistors, capacitors and inductors represent hydraulic resistance, compliance and inertial effects, respectively. Arrows represent variable circuit elements. The four main interconnected compartments are as follows: (i) the heart 12, where the valves are represented by ideal switches and the ventricular pumps are modeled by means of voltage sources; (ii) the systemic circulation 14, where the arterial segments from the ascending aorta to the iliac arteries are modeled in great detail because of their relevance to the BCG signal; (iii) the pulmonary circulation 16, which starts with the pulmonary artery at the right ventricle and converges into the left atrium; and (iv) the cerebral circulation 18, which branches from the aortic arch and converges into the systemic venous system.

In FIG. 1, circuit nodes have been marked by thick black dots and numeric labels from 1 to 14 enclosed in circles or squares to distinguish between the four circulatory compartments included in the model (heart, systemic circulation, pulmonary circulation, cerebral circulation, respectively). In particular, nodes 1-6, 10, 11 and 14 were utilized for the computation of the BCG signal. Arterial pressure and volume waveforms simulated via the closed-loop model were also utilized.

Table I illustrates the anatomical meaning of the circuit nodes 1-14 with respect to the four circulatory compartments included in the model (heart, systemic circulation, pulmonary circulation, cerebral circulation) as shown in FIG. 1. Cerebral capillaries are assumed to be non-compliant as is conventionally understood, and as such node 15 in Table I, which corresponds to cerebral veins, is not represented in FIG. 1.

TABLE I
ANATOMICAL MEANING OF THE CIRCUIT NODES

| NODE | COMPARTMENT | SEGMENT | y [CM] |
|---|---|---|---|
| 1 | Heart | Left Ventricle | 0.5 |
| 2 | Systemic | Ascending Aorta | −2 |
| 3 | Systemic | Aortic Arch | −7 |
| 4 | Systemic | Thoracic Aorta | 20 |
| 5 | Systemic | Abdominal Aorta | 35 |
| 6 | Systemic | Iliac arteries | 45 |
| 7 | Systemic | Small arteries | — |
| 8 | Systemic | Capillaries | — |
| 9 | Systemic | Veins | — |
| 10 | Heart | Right Ventricle | 0.5 |
| 11 | Pulmonary | Pulmonary Arteries | −5 |
| 12 | Pulmonary | Capillaries | — |
| 13 | Pulmonary | Veins | — |
| 14 | Cerebral | Cerebral Arteries | −10 |
| 15 | Cerebral | Veins | — |

In the model 10, resistors, capacitors and inductors represent hydraulic resistance, wall compliance and inertial effects, respectively. Variable capacitors, indicated with arrows in FIG. 1, are utilized to describe the wall viscoelasticity in large arteries and the nonlinear properties of the heart muscle fibers. The four heart valves are modeled as ideal switches. The ventricular pumps are modeled as time-dependent voltage sources. The model 10 leads to a system of ordinary differential equations (ODEs), whose solution provides the time-dependent profiles of pressures and volumes at the circuit nodes and flow rates through the circuit branches, wherein the closed-loop model 10 depicted in FIG. 1 can be represented mathematically by ODEs of the form shown in Equation (5):

$$ℳ ( Y ( t ) ) \frac{dY ( t )}{dt} = 𝒜 ( Y ( t ) ) Y ( t ) + b ( Y ( t ) ) t ∈ [ 0 , T ] \qquad ( 5 )$$

In Equation (5), the specific expressions for Y, [symbol] , [symbol] and b follow from constitutive equations characterizing the circuit elements and the Kirchoff laws of currents and voltages.

The experimental methodology included utilizing the closed-loop model 10 to simulate the BCG signal by substituting in Equations (2) through (4) the volume waveforms computed via the solution of the mathematical model. Nine contributions were included in the calculations for the BCG signal, meaning that N=9, which accounts for the volume waveforms pertaining to the left and right ventricles (nodes 1 and 10), four aortic segments (nodes 2, 3, 4, 5), iliac arteries (node 6), pulmonary arteries (node 11) and cerebral arteries (node 14). The circuit nodes whose volume waveforms are included in the BCG are surrounded with square frames in FIG. 1. The calculation of the BCG signal required the values of they-coordinate representing the distance of each compartment of interest from an ideal plane through the heart 12, as depicted in FIG. 1, with the node values used for experimentation being summarized in Table I.

While the mathematical model 10 was simulated in Open-Modelica software (an open-source Modelica-based modeling and simulation environment intended for industrial and academic studies of complex dynamic systems), any capable software or programming language (e.g., Python) can be used. Model results were obtained using a differential algebraic system solver (DASSL), with a tolerance of 10⁻⁶ and a time step of 0.001 s. Eight cardiac cycles were simulated, for a total simulation time of 6.4 s, in order to obtain a periodic solution. While simulation results were post-processed using Matlab (a commercial software to analyze data, develop algorithms and create mathematical models), use of any similar capable post-processing software is envisioned.

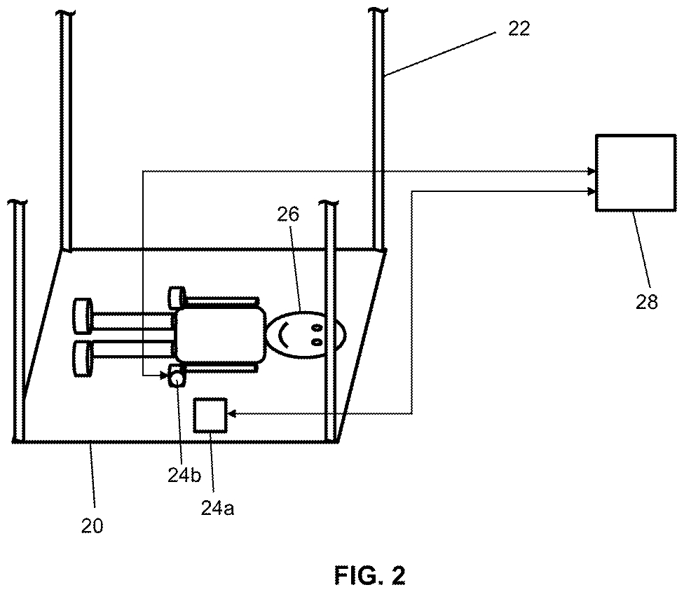

The results from the experimental tests performed correspond to the eight simulated cardiac cycles. As shown in FIG. 2, the experimental tests included using a bed 20 suspended with suspension cables 22 and equipped with a three-axis accelerometer 24a (such as from Kionix Inc.) with a sensitivity of 1000 mV/g within the range of ±2 g. The accelerometer 24a can be placed at any location on the bed 20 where the accelerometer 24a is capable of measuring the forces (e.g., underneath the bed 20). A healthy test subject 26 (e.g., in one test scenario comprising a male subject of age 33, weight 78 kg and height 178 cm), laid on the bed 20 in the supine position for approximately 10 minutes. A pulse sensor 24b was wrapped around the ring finger of the subject's left hand. Three-axis accelerometer signals from accelerometer 24a as well as the signal from the pulse sensor 24b were collected simultaneously using a data acquisition system 28 (such as an AD Instruments PowerLab Data Acquisition system) at a rate of 1000 samples per second (however, usage of other acquisition systems is envisioned). Local peaks of the pulse waveforms were used as the reference for heart beat activities, and segmentation of the BCG waveforms. Beats of length larger than 1.5 seconds or less than 0.4 seconds, corresponding to 40 and 150 beats per minute (bpm), respectively, were considered abnormal and were removed in pre-processing. The main focus is the y-axis signal of the accelerometer since it provides the experimental waveform of BCG_{acc}(t) defined in Equation (4). A band-pass Butterworth filter with cut-off frequencies at 0.7 Hz to 15 Hz was applied to eliminate low frequency respiratory base line and high frequency noises. The resulting acceleration signal was segmented based on the timings previously acquired from the pulse sensor 24b.

The data acquired from the subjects was processed by processing hardware in order to extract a representative BCG waveform over a cardiac cycle. The data was also scanned for outliers. Since the heart rate is naturally subject to variations, the length of each BCG cycle was not constant over the approximately 10 minutes of data acquisition. Thus, after removal of the outliers, the signals were cut and re-sampled to the median length of all, which resulted in 83 bpm for the subject under consideration. For ease of comparison with conventional displacement BCG data (e.g., based on a cardiac cycle of 0.8 seconds, corresponding to 75 bpm), the collected data was re-sampled to match the length of 0.8 seconds for the cardiac cycle. In order to further remove certain potential effects due to respiratory movements, the mean was subtracted from each waveform, thereby making it zero-mean. Then, all waveforms were aligned to the median one, based on their cross-correlation value. All waveforms with correlation below 0.4 and lag-time above 0.4 seconds were considered to be motion artifacts and removed from the analysis. The signals for velocity and displacement BCGs were obtained by integration starting from the acceleration waveform.

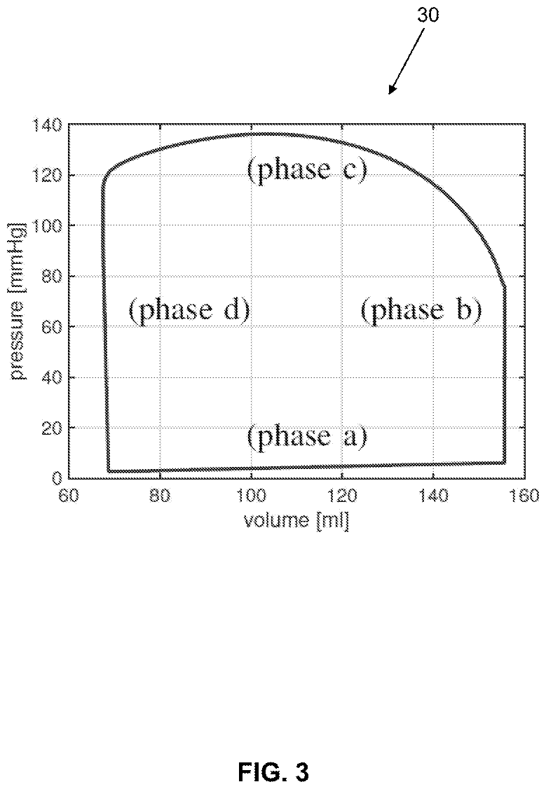

FIG. 3 illustrates a plot 30 of a qualitative description of left ventricular function, which reports the volume-pressure relationship in the left ventricle during one cardiac cycle simulated via the closed-loop model 10. Results indicated that the closed-loop model 10 correctly captured the four basic phases of ventricular function: ventricular filling (phase a in FIG. 3), isovolumetric contraction (phase b in FIG. 3), ejection (phase c in FIG. 3), and isovolumetric relaxation (phase d in FIG. 3).

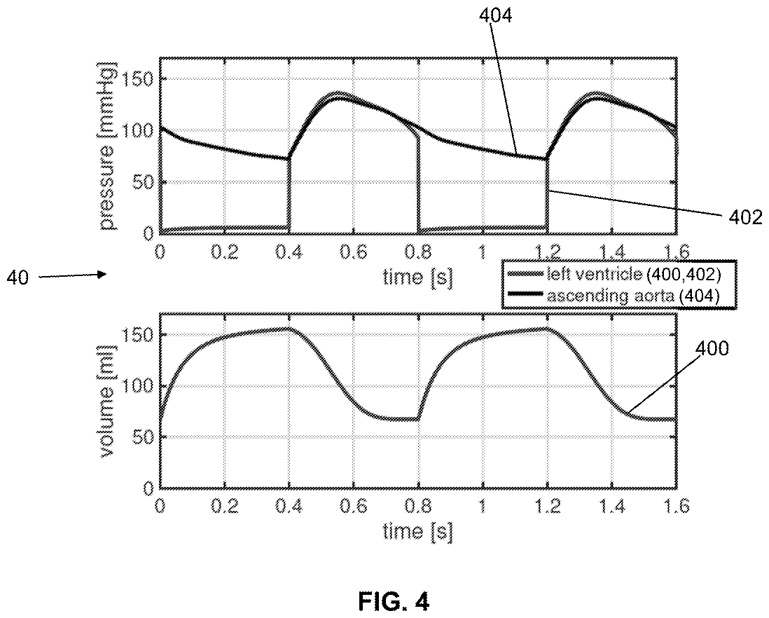

FIG. 4 illustrates another qualitative representation of ventricular function, as provided by a Wiggers diagram 40, wherein the x-axis is used to plot time, while the y-axis contains all of the following on a single grid: blood pressure; aortic pressure; ventricular pressure; atrial pressure; ventricular volume; electrocardiogram; arterial flow (optional); and heart sounds (optional). The Wiggers diagram 40 clearly illustrates the coordinated variation of these values as the heart beats, assisting in the understanding of the entire cardiac cycle.

As illustrated in FIG. 4, the volume waveform 400 pertaining to the left ventricle is portrayed together with the pressure waveforms pertaining to the left ventricle 402 and the ascending aorta 404. The Wiggers diagram 40 simulated via the closed-loop model 10 indicated the typical features of isovolumetric contraction and relaxation exhibited by physiological waveforms.

Quantitative parameters describing cardiovascular physiology include end-diastolic volume (EDV), end-systolic volume (ESV), stroke volume (SV), cardiac output (CO) and ejection fraction (EF) associated with the left and right ventricles. EDV and ESV are computed as the maximum and minimum values of the ventricular volumes during the cardiac cycle, respectively, and their difference gives SV, namely SV=EDV−ESV. The relative difference between EDV and ESV gives EF, namely EF=100×(EDV−ESV)/EDV, which can also be written as EF=100×SV/EDV. Then, denoting by T_{c} the length of the heart beat measured in seconds, the heart rate HR and the cardiac output CO are computed as HR=60/T_{c} and CO=HR×SV/1000.

Table II shows the values of these parameters for the left and right ventricle as known in the field and as simulated via the closed-loop model 10.

TABLE II
CARDIOVASCULAR PHYSIOLOGY: LEFT AND RIGHT VENTRICLES

| | | NORMAL CLINICAL RANGE | | CLOSED-LOOP MODEL | |
|---|---|---|---|---|---|
| | | Left | Right | Left | Right |
| PARAMETER | UNIT | Ventricle | Ventricle | Ventricle | Ventricle |
| End-Diastolic | [ml] | 142 (102, 183) | 144 (98, 190) | 157.5 | 154.1 |
| Volume (EDV) | | | 100-160 | | |
| End-Systolic | [ml] | 47 (27, 68) | 50 (22, 78) | 68.6 | 65.0 |
| Volume (ESV) | | 50-100 | | | |
| Stroke | [ml/beat] | 95 (67, 123) | 94 (64, 124) | 88.9 | 89.1 |
| Voiume (SV) | | 60-100 | 60-100 | | |
| Cardiac | [l/min] | 4-8 | 4-8 | 6.7 | 6.7 |
| Output (CO) | | | | | |
| Ejection | [%] | 67 (58, 76) | 66 (54, 78) | 56.5 | 57.8 |
| Fraction (FE) | | | 40-60 | | |

Known studies in the field utilized cardiovascular magnetic resonance to assess left and right ventricular functions on (e.g., 120) healthy individuals. All the simulated values of the model 10 fall within the ranges reported in these known studies, thereby validating the capability of the closed-loop model 10 to capture the main features of the heart functions. The pressure and volume waveforms pertaining to the main segments of the systemic arteries simulated via the closed-loop model 10 are shown in FIG. 1. The results indicated that the model 10 captures the typical features of peak magnification and time delay exhibited by physiological waveforms known in the field.

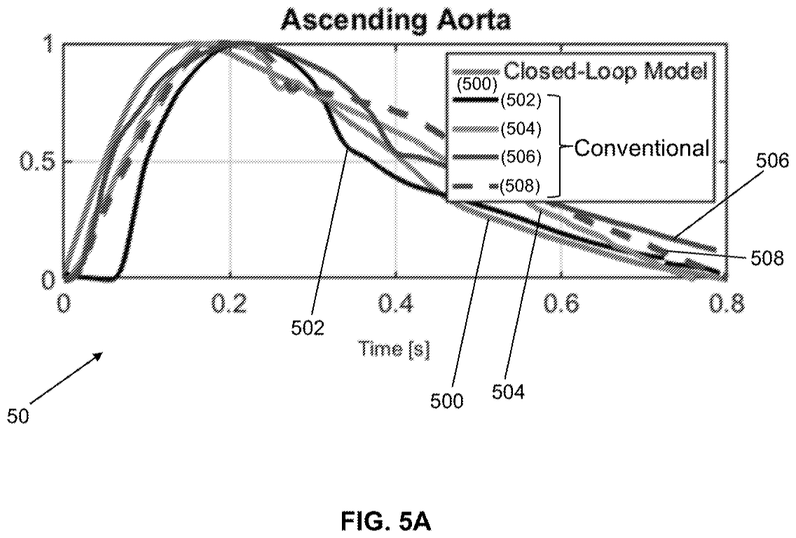

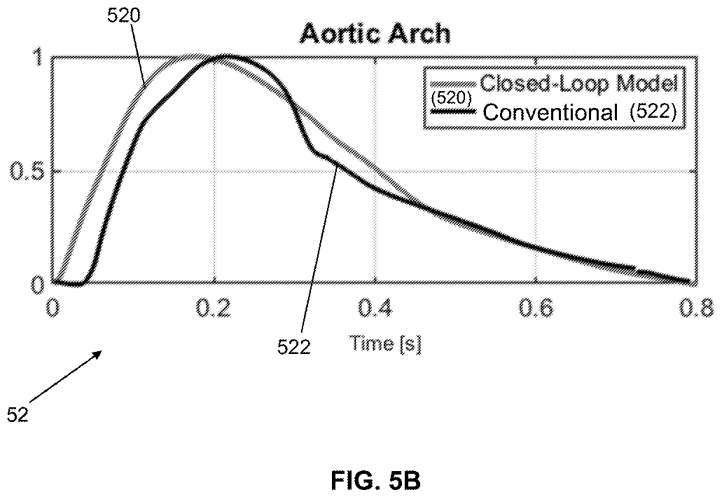

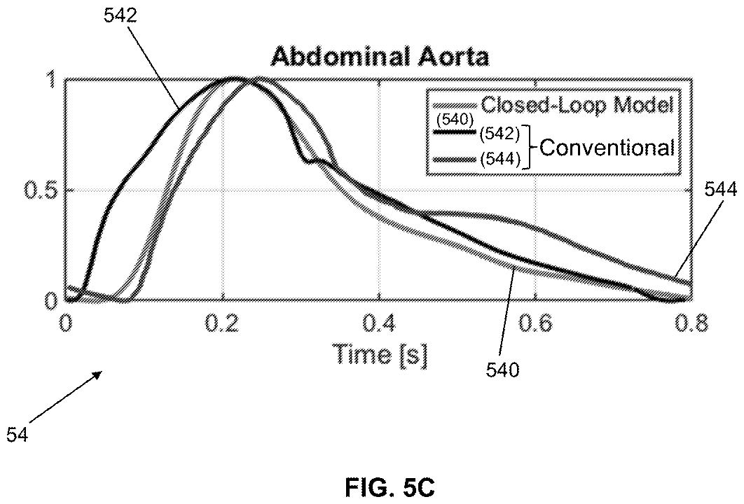

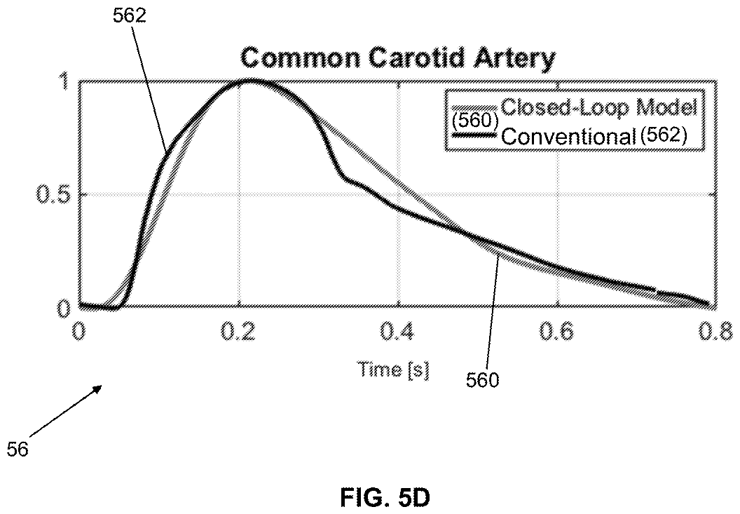

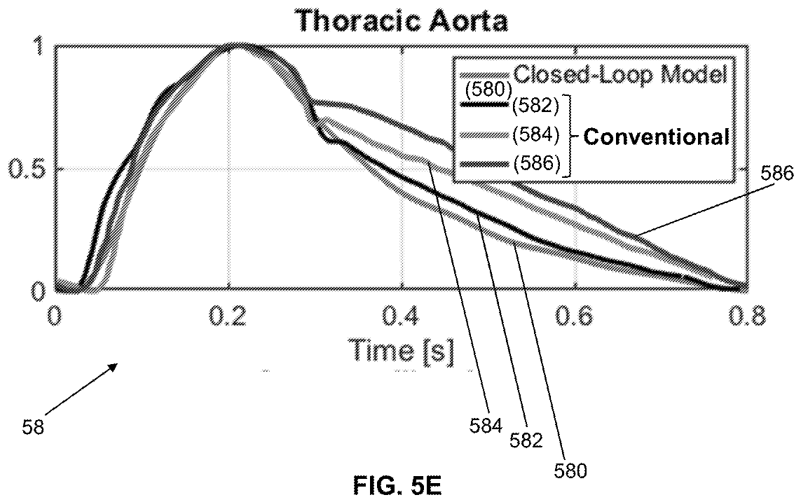

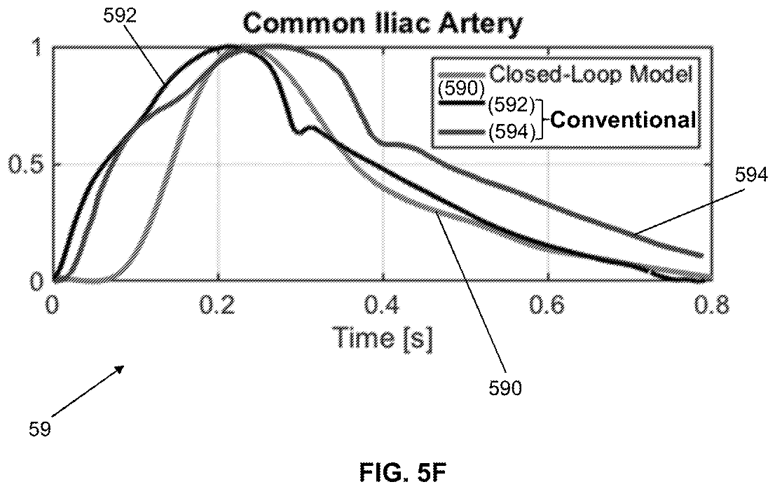

FIGS. 5A to 5F illustrate pressure waveforms simulated via the closed-loop model 10 of the present application compared with (i) experimental measurements at different sites along the arterial tree as is known in the art; and (ii) theoretical simulations generated via a one-dimensional arterial network as is known in the art. In particular, the results of simulations of the present model 10 were compared with conventional results known in the field, where sensor tipped intra-arterial wires were used to measure pressure in various (e.g., ˜20 subjects, age 35-73 years) at 10-cm intervals along the aorta, starting at the aortic root; (ii) where aortic pressures at 10, 20, 30, 40, 50 and 60 cm downstream from the aortic valve was reported; and (iii) where arterial waveforms were generated via a one-dimensional arterial network. For ease of comparison, all the waveforms were normalized to a unitary amplitude and were resealed in time to last 0.8 s.

FIG. 5A shows an ascending aorta comparison 50 comparing the waveform 500 of the model 10 with conventional waveforms 502, 504, 506 and 508, FIG. 5B shows an aortic arch comparison 52 comparing the waveform 520 of the model 10 with conventional waveform 522, FIG. 5C shows an abdominal aorta comparison 54 comparing waveform 540 of model 10 with conventional waveforms 542 and 544, FIG. 5D shows a common carotid aorta comparison 56 comparing waveform 560 of model 10 to conventional waveform 562, FIG. 5E shows a thoracic aorta comparison 58 comparing waveform 580 of model 10 and conventional waveforms 582, 584 and 586, and FIG. 5F shows a common iliac artery comparison 59 comparing waveform 590 of model 10 and conventional waveforms 592 and 594. As shown, FIGS. 5A to 5F exhibit close agreement between the theoretical and experimental waveforms, and the results shown in FIGS. 5A to 5F illustrate the capability of the present closed-loop model 10 to capture prominent features of cardiovascular physiology and supports the utilization of the model output to construct a theoretical BCG.

The volume waveforms simulated using the closed-loop model 10 in FIG. 1 were utilized in Equation (2) to calculate, theoretically, the waveform BCG_{disp}(t) associated with the displacement of the center of mass. The BCG waveforms for velocity and acceleration, namely BCGᵥₑₗ(t) and BCG_{acc}(t), are obtained from BCG_{disp}(t) via discrete time-differentiation. Following conventions in the art, the BCG waveforms are reported by way of the auxiliary functions f_{D}, f_{V} and f_{A} defined as:

$${f}_{D} ( t ) \text{:=} M \cdot {BCG}_{disp} ( t ) = {ρ}_{b} {\sum }_{i = 1}^{N} {V}_{i} ( t ) {y}_{i} [ g cm ]$$

$${f}_{V} ( t ) \text{:=} M \cdot {BCG}_{vel} ( t ) = {ρ}_{b} {\sum }_{i = 1}^{N} \frac{{dV}_{i} ( t )}{dt} {y}_{i} [ g cm {s}^{- 1} ]$$

$${f}_{A} ( t ) \text{:=} M \cdot {BCG}_{acc} ( t ) = {ρ}_{b} {\sum }_{i = 1}^{N} \frac{{d}^{2} {V}_{i} ( t )}{{dt}^{2}} {y}_{i} [ dyne ]$$

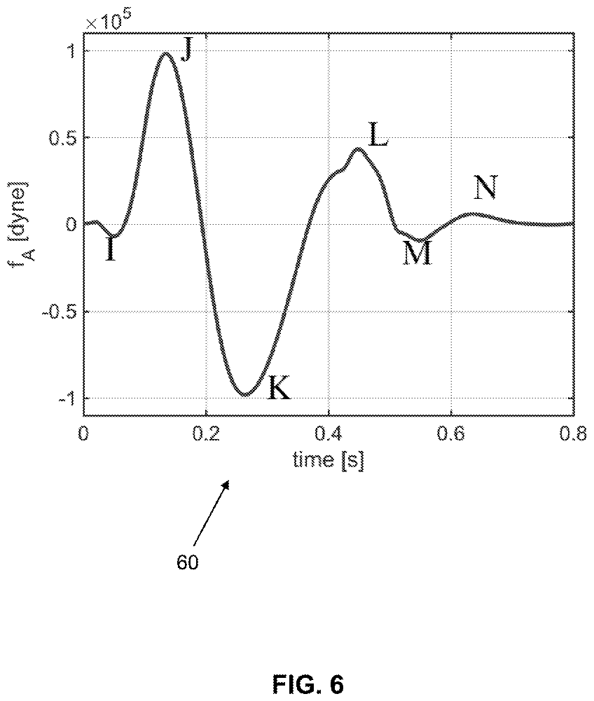

FIG. 6 illustrates the waveform f_{A}(t) 60 simulated via the closed-loop model 10 over one cardiac cycle. The waveform exhibits the typical I, J, K, L, M and N peaks and valleys that characterize BCG signals as measured experimentally in the art, thereby confirming the capability of the closed-loop model 10 to capture the fundamental cardiovascular mechanisms that give rise to the BCG signal.

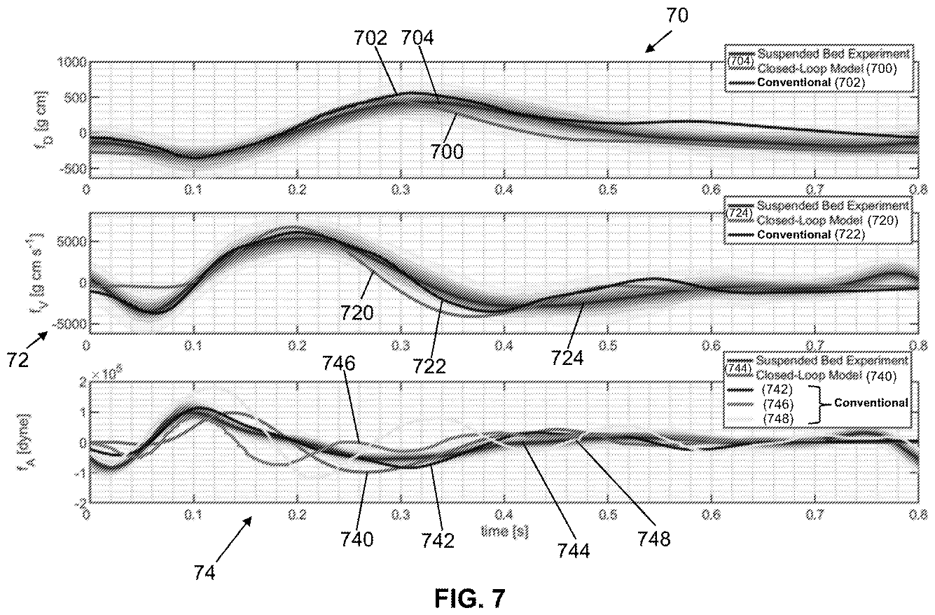

FIG. 7 illustrates BCG waveforms for displacement (top plot 70), velocity (middle plot 72) and acceleration (bottom plot 74), denoted by f_{D}, f_{V} and f_{A}, respectively. More specifically, FIG. 7 shows the waveforms 700, 720 and 740 simulated via the closed-loop model 10, the conventional waveforms 702, 722, 742, 746 and 748 calculated in the art, and the experimental waveforms 704, 724 and 744 obtained in the suspended bed experiment illustrated in FIG. 2. A quantitative comparison between the simulated BCG waveforms pertaining to displacement, velocity and acceleration of the center of mass shown in FIG. 7 was performed by use of the auxiliary functions f_{D}, f_{V} and f_{A}. The waveforms simulated via the closed-loop model 10 are compared with (i) the theoretical results in the art, where the inventors calculated velocity and acceleration waveforms via time-differentiation of the reported signal for the displacement; (ii) the experimental results obtained in the art utilizing a modified weighing scale; (iii) the experimental results obtained in the art utilizing direct pressure measurements; and (iv) the experimental results obtained in the suspended bed experiment (e.g., utilizing an accelerometer 24a on a suspended bed 20 as shown in FIG. 2), where a mass M=87 kg of the study subject was utilized to calculate f_{A}. A time-shift was applied in order to align the waveforms for ease of comparison. The results in FIG. 7 confirm that the BCG waveforms simulated via the closed-loop model 10 were of the same order of magnitude as those reported in previous theoretical and experimental studies for all three aspects of the body motion, namely displacement, velocity and acceleration. FIG. 7 also illustrates that theoretical and experimental BCG waveforms exhibit similar trends, typically marked by the peaks shown in FIG. 6, but differ in the precise timing of these peaks and in their magnitude. Such differences are likely attributable to physiological variability of cardiovascular parameters among individuals, for example associated with age, gender and ethnicity, and to different techniques utilized to acquire the BCG signal, such as a modified weighing scale and the accelerometer on the suspended bed.

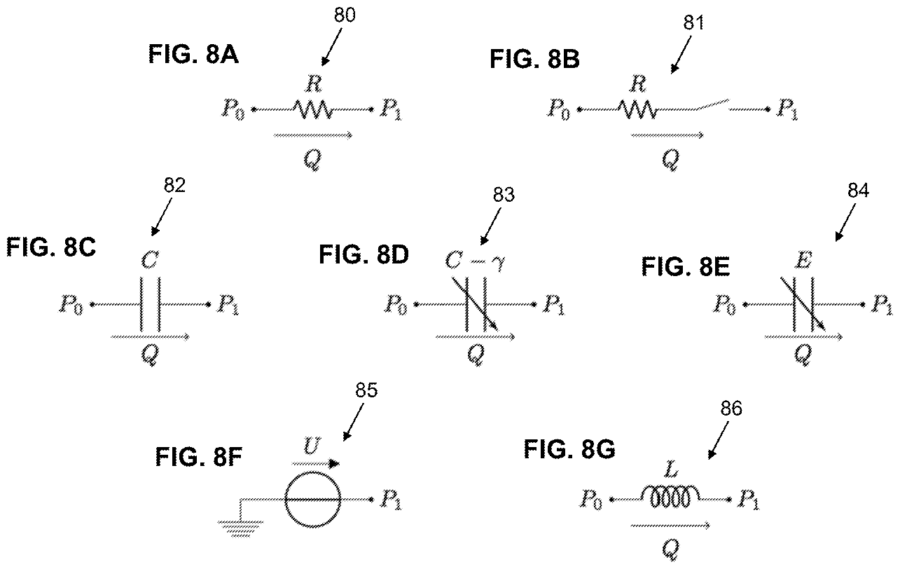

FIGS. 8A to 8G illustrate electrical/circuit elements included in the closed-loop model 10. The constitutive laws characterizing each element are detailed below.

FIG. 8A illustrates a linear resistor 80, where the hydraulic analog to Ohm's law states that the pressure difference P₀−P₁ across the resistor is proportional to the volumetric flow rate Q, with the hydraulic resistance R as proportionality constant as shown in Equation (6a):

P₀−P₁=RQ (6a)

FIG. 8B illustrates a linear resistor with ideal switch 81, used to model heart valves. The switch is completely closed as soon as the pressure difference P₀−P₁ is positive and completely open otherwise, as shown in Equation (6b):

$$Q = {σ}_{{P}_{0} - {P}_{1}} \frac{{P}_{0} - {P}_{1}}{R} \qquad ( 6 b )$$

where σ_{P0-P1} is a binary-valued function of the pressure pair P₀, P₁ defined in Equation (6c) as:

$${σ}_{{P}_{0} - {P}_{1}} \text{:=} \{ \begin{matrix}1 & if {P}_{0} - {P}_{1} > 0 \\ 0 & otherwise \end{matrix} \qquad ( 6 c )$$

FIG. 8C illustrates a linear capacitor 82. In a capacitor, the time rate of change of the fluid volume V stored in the capacitor equals the volumetric flow rate Q, as shown in Equation (6d):

$$\frac{dV}{dt} = Q \qquad ( 6 d )$$

In the case of a linear capacitor, the volume V and the pressure difference P₀−P₁ are related by a proportionality law as shown in Equation (6e), where C is a positive constant:

V=C(P₀−P₁) (6e)

FIG. 8D illustrates a variable capacitor 83 for arterial viscoelasticity. The law for the capacitor stated by Equation (6d) is coupled with the following differential relationship between the pressure difference P₀−P₁ and the fluid volume V as shown in Equation (6f), where C and γ are positive constants. Equation (6f) corresponds to adopting a linear viscoelastic thin shell model for the arterial walls:

$${P}_{0} - {P}_{1} = \frac{V}{C} + γ \frac{dV}{dt} \qquad ( 6 f )$$

FIG. 8E illustrates a variable capacitor 84 for ventricular elastance, where the law for the capacitor stated by Equation (6d) is coupled with the following relationship between the pressure difference P₀−P₁ and the fluid volume V:

P₀−P₁=E(t)V (6g)

where E(t) is a given function of time modeling the complex biomechanical properties of the ventricular wall, and it was assumed that E_{L}(t)=ELD+ELS a_{L}(t) and E_{R}(t)=ERD+ERS a_{R}(t), where ELD, ELS, ERD and ERS are given constants and a_{L}(t)=(tan h(q_{L}(Tₘ−Tₐ)−tan h(q_{L}(Tₘ−T_{b})))/2 and a_{R}(t)=(tan h(q_{R}(Tₘ−Tₐ)−tan h(q_{R}(Tₘ−T_{b})))/2 if Tₘ<Tₛ and a_{L}(t)=a_{R}(t)=0 otherwise. Here, Tₘ is defined as Tₘ=mod (t, T_{c}), where Tₛ and T_{c} are the systolic and cardiac periods, respectively, and Tₐ and T_{b} are given constants.

FIG. 8F illustrates a voltage source 85, where the hydraulic analog of a voltage source is an element that imposes the nodal pressure as in Equation (6h):

P₁(t)=U(t) (6h)

where U(t) is a given function, and it was assumed that U_{L}(t)=ULOa_{L}(t) and U_{R}(t)=UROa_{R}(t), where ULO and URO are positive constants and the functions a_{L}(t) and a_{R}(t) are the same as described above.

FIG. 8G illustrates a linear inductor 86, where the time rate of change of the volumetric flow rate Q is related to the pressure difference P₀−P₁ by the following proportionality law shown in Equation (6i), where L is a positive constant:

$$L \frac{dQ}{dt} = {P}_{0} - {P}_{1} \qquad ( 6 i )$$

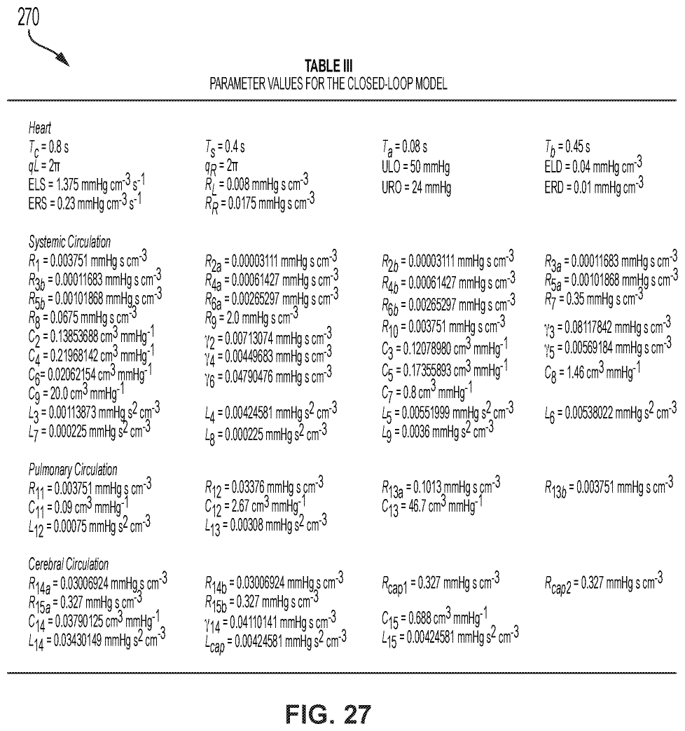

The parameter values 270 for each of the circuit elements are shown in Table III of FIG. 27. Regarding Table III, the parameter values pertaining to the heart, the systemic microcirculation and the pulmonary circulation were adapted from what is known in the art, but are able to be adapted to be reflective of particular parameters of CVD, an individual patient, or other cardiovascular biomarkers. The parameter values pertaining to the main arteries were computed using the following constitutive equations:

$$R = \frac{8 π l η}{{S}^{2}} L = \frac{{ρ}_{b} l}{S} C = \frac{3 {lS ( a + 1 )}^{2}}{E ( 2 a + 1 )} γ = \frac{δ}{C}$$

where a=r/h is the ratio between vessel radius r and wall thickness h, l is the vessel length, S=πr² is the vessel cross-sectional area, ρ_{b} is the blood density, η is the blood viscosity, E and δ are the Young modulus and the viscoelastic parameter characterizing the vessel wall. It was assumed ρ_{b}=1.05 g cm⁻³, η=0.035 g cm⁻¹ s⁻¹, E=4·10⁶ dyne cm⁻² and δ=9.81·10⁻³ s. The values of the remaining geometrical parameters utilized to determine R, L, C, Cᵥ and γ for each of the main arterial segments were adapted from what is known, as shown in Table IV:

TABLE IV
GEOMETRICAL PARAMETERS FOR
THE MAIN ARTERIAL SEGMENTS

| ARTERIAL SEGMENT | l [cm] | r [cm] | h [cm] |
|---|---|---|---|
| Ascending Aorta | 4 | 1.44 | 0.158 |
| Aortic Arch | 5.9 | 1.14 | 0.134 |
| Thoracic Aorta | 15.6 | 0.96 | 0.117 |
| Abdominal Aorta | 15.9 | 0.85 | 0.105 |
| Iliac artery | 5.8 | 0.52 | 0.076 |

The vector Y(t) of the circuit unknowns in Equation (5) is defined as the column vector in Equation (7):

Y(t)=[ [symbol] (t); [symbol] (t)]^{T} (7)

where the two row vectors [symbol] and [symbol] are defined as:

[symbol]

(t)=[V_{L}(t),V₂(t),V₃(t),V₄(t),V₅(t),V₆(t),V₇(t),V₈(t),V₉(t),V_{R}(t),V₁₁(t),V₁₂(t),V₁₃(t),V₁₄(t),V₁₅(t)]

[symbol]

(t)=[Q₃(t),Q₄(t),Q₅(t),Q₆(t),Q₇(t),Q₈(t),Q₉(t),Q₁₂(t),Q₁₃(t),Q₁₄(t),Q₁₅(t)].

The symbols V_{L} and V_{R} denote the fluid volume stored in the variable capacitors for ventricular elastance characterized by E_{L} and E_{R}, respectively; the symbols Vᵢ, i=2, . . . , 6 and i=14 denote the fluid volume stored in the variable capacitors for arterial viscoelasticity characterized by Cᵢ and i; the symbols Vᵢ, i=7, . . . , 9 and i=15 denote the fluid volume stored in the linear capacitors characterized by Cᵢ, the symbols Qᵢ, i=3, . . . , 9 and i=12, . . . , 15, denote the volumetric flow rate through the inductor characterized by Lᵢ. To derive the nonlinear system of ODEs of Equation (5) representing the mathematical model of blood circulation in the human body, the following three steps were taken: 1) Kirchoff's current laws (KCLs) were written for each of the nodes marked on the circuit in FIG. 1; 2) Kirchoff s voltage laws (KVLs) were written for each of the 11 circuit branches containing an inductor; 3) the Equations (6a) to (6i) were substituted in the KCLs and KVLs. The system was solved using the initial conditions shown in Table V:

TABLE V
INITIAL CONDITIONS FOR THE TIME-
DEPENDENT MODEL SIMULATIONS

| VARIABLE | VALUE | VARIABLE | VALUE |
|---|---|---|---|
| V_{L} | 71.2700 cm³ | Q₃ | 1.68 cm³ s⁻¹ |
| V₂ | 73.5486 cm³ | Q₄ | 1.9961 cm³ s⁻¹ |
| V₃ | 71.9746 cm³ | Q₅ | 1.1861 cm³ s⁻¹ |
| V₄ | 71.9983 cm3 | Q₆ | 9.03697 cm³ s⁻¹ |
| V₅ | 71.9327 cm³ | Q₇ | 17.8121 cm³ s⁻¹ |
| V₆ | 72.2213 cm³ | Q₈ | 19.1462 cm³ s⁻¹ |
| V₇ | 80.9077 cm³ | Q₉ | 67.359 cm³ s⁻¹ |
| V₈ | 70.537 cm³ | Q₁₂ | 0.7861 cm³ s⁻¹ |
| V₉ | 3.3268 cm³ | Q₁₃ | 23.83 cm³ s⁻¹ |
| V_{R} | 3.1638 cm³ | Q₁₄ | 0.5909 cm³ s⁻¹ |
| V₁₁ | 13.4160 cm³ | Q₁₅ | 1.9961 cm³ s⁻¹ |
| V₁₂ | 13.3920 cm³ | | |
| V₁₃ | 11.2950 cm³ | | |
| V₁₄ | 70.9869 cm³ | | |
| V₁₅ | 3.3268 cm³ | | |

Simulations were run until a periodic solution was established.

With further reference to Equation (5), overall, the differential system in Equation (5) includes m=26 differential equations. The expressions of the nonzero entries of the matrices [symbol] and [symbol] as well as of the forcing vector b are shown below. Let:

\tilde{R}_{L}:=R_{L}+R₁+R₂ₐ,

\tilde{R}_{R}:=R_{R}+R₁₁,

\tilde{R}_{cap}:=R_{14b}+R_{cap1}+R_{cap2}+R₁₅ₐ and

\tilde{L}_{cap}:=L_{cap}+L₁₅.

The nonzero entries of [symbol] are:

$${ℳ}_{1 , 1} = 1 , {ℳ}_{12} = - \frac{{γ}_{2}}{{\tilde{R}}_{L}} {σ}_{{P}_{1} - {P}_{2}}$$

$${ℳ}_{2 , 2} = 1 + \frac{{γ}_{2}}{{\tilde{R}}_{L}} {σ}_{{P}_{1} - {P}_{2}}$$

$${ℳ}_{i , i} = 1 i = 3 , … , 15$$

$${ℳ}_{i , j} = {γ}_{j} , {ℳ}_{i , j + 1} = - {γ}_{j + 1} , {ℳ}_{i , i} = - {L}_{j + 1} , \\ i = 16 , … , 19 , j = i - 14 , \\ {ℳ}_{20 , 6} = {γ}_{6} , {ℳ}_{20 , 20} = - {L}_{7}$$

$${ℳ}_{i , i} = - {L}_{k} , i = 21 , 22 , k = i - 13 , \\ {ℳ}_{i , i} = - {L}_{k} , i = 23 , 24 , k = i - 11 , \\ {ℳ}_{25 , 3} = {γ}_{3} , {ℳ}_{25 , 14} = - {γ}_{14} , {ℳ}_{25 , 25} = - {L}_{14}$$

$${ℳ}_{26 , 4} = {γ}_{14} , {ℳ}_{26 , 26} = - {\tilde{L}}_{cap} .$$

The nonzero entries of [symbol] are:

$${𝒜}_{1 , 1} = - {E}_{L} ( \frac{{σ}_{{P}_{13} - {P}_{1}}}{{R}_{13 b}} + \frac{{σ}_{{P}_{1} - {P}_{2}}}{{\tilde{R}}_{L}} )$$

$${𝒜}_{1 , 2} = \frac{{σ}_{{P}_{1} - {P}_{2}}}{{\tilde{R}}_{L} {C}_{2}} , {𝒜}_{1 , 13} = \frac{{σ}_{{P}_{13} - {P}_{1}}}{{R}_{13 b} {C}_{13}}$$

$${𝒜}_{2 , 1} = {E}_{L} \frac{{σ}_{{P}_{1} - {P}_{2}}}{{\tilde{R}}_{L}} , {𝒜}_{2 , 2} = - \frac{{σ}_{{P}_{1} - {P}_{2}}}{{\tilde{R}}_{L} {C}_{2}} , {𝒜}_{2 , 16} = - 1$$

$${𝒜}_{3 , 16} = 1 , {𝒜}_{3 , 17} = - 1 , {𝒜}_{3 , 25} = - 1$$

$${𝒜}_{i , j} = 1 , {𝒜}_{1 , j + 1} = - 1 i = 4 , … , 8 , j = i + 13 , \\ {𝒜}_{9 , 9} = - \frac{1}{{C}_{98}} ( \frac{{σ}_{{P}_{9} - {P}_{10}}}{{R}_{10}} + \frac{1}{{R}_{15 b}} )$$

$${𝒜}_{9 , 10} = \frac{{E}_{R}}{{R}_{10}} {σ}_{{P}_{9} - {P}_{10}} , {𝒜}_{9 , 15} = \frac{1}{{R}_{15 b} {C}_{15}} , {𝒜}_{9 , 22} = 1$$

$${𝒜}_{10 , 9} = \frac{{σ}_{{P}_{9} - {P}_{10}}}{{R}_{10} {C}_{9}} , {𝒜}_{10 , 10} = - {E}_{R} ( \frac{{σ}_{{P}_{9} - {P}_{10}}}{{R}_{10}} + \frac{{σ}_{{P}_{10} - {P}_{11}}}{{\tilde{R}}_{R}} )$$

$${𝒜}_{10 , 11} = \frac{{σ}_{{P}_{10} - {P}_{11}}}{{\tilde{R}}_{R} {C}_{11}} , {𝒜}_{11 , 10} = {E}_{R} \frac{{σ}_{{P}_{10} - {P}_{11}}}{{\tilde{R}}_{R}} , \\ {𝒜}_{11 , 11} = - \frac{{σ}_{{P}_{10} - {P}_{11}}}{{\tilde{R}}_{R} {C}_{11}} , {𝒜}_{11 , 23} = - 1 , {𝒜}_{12 , 23} = 1 , \\ {𝒜}_{12 , 24} = - 1 , {𝒜}_{13 , 1} = {E}_{L} \frac{{σ}_{{P}_{13} - {P}_{1}}}{{R}_{13 b}} , {𝒜}_{13 , 13} = - \frac{{σ}_{{P}_{13} - {P}_{1}}}{{R}_{13 b} {C}_{13}} , \\ {𝒜}_{13 , 24} = 1 , {𝒜}_{14 , 25} = 1 , {𝒜}_{14 , 26} = - 1$$

$${𝒜}_{15 , 9} = \frac{1}{{R}_{15 b} {C}_{9}} , {𝒜}_{15 , 15} = - \frac{1}{{R}_{15 b} {C}_{15}} , {𝒜}_{15 , 26} = 1$$

$${𝒜}_{i , j} = - \frac{1}{{C}_{j}} , {𝒜}_{i , j + 1} = \frac{1}{{C}_{j + 1}} , {𝒜}_{i , i} = ( {R}_{jb} + {R}_{j + 1 a} ) , \\ i = 16 , … , 29 , j = i - 14 , \\ {𝒜}_{20 , 6} = - \frac{1}{{C}_{6}} , {𝒜}_{20 , 7} = \frac{1}{{C}_{7}} , {𝒜}_{20 , 20} = ( {R}_{7} + {R}_{6 b} )$$

$${𝒜}_{i , j} = - \frac{1}{{C}_{j}} , {𝒜}_{i , j + 1} = \frac{1}{{C}_{j + 1}} {𝒜}_{i , i} = {R}_{j + 1} , \\ i = 21 , 22 , j = i - 14$$

$${𝒜}_{23 , 11} = - \frac{1}{{C}_{11}} , {𝒜}_{23 , 12} = \frac{1}{{C}_{12}} , {𝒜}_{23 , 23} = {R}_{12}$$

$${𝒜}_{24 , 12} = - \frac{1}{{C}_{12}} , {𝒜}_{24 , 13} = \frac{1}{{C}_{13}} , {𝒜}_{24 , 24} = {R}_{13 a}$$

$${𝒜}_{25 , 3} = - \frac{1}{{C}_{3}} , {𝒜}_{25 , 14} = \frac{1}{{C}_{14}} , {𝒜}_{25 , 25} = {R}_{14 a}$$

$${𝒜}_{26 , 14} = - \frac{1}{{C}_{14}} , {𝒜}_{26 , 15} = \frac{1}{{C}_{15}} , {𝒜}_{26 , 26} = {\tilde{R}}_{cap} .$$

The nonzero entries of the forcing term b are:

$${b}_{1} = - {U}_{L} ( \frac{{σ}_{{P}_{13} - {P}_{1}}}{{R}_{13 b}} + \frac{{σ}_{{P}_{1} - {P}_{2}}}{{\tilde{R}}_{L}} )$$

$${b}_{2} = \frac{{U}_{L}}{{\tilde{R}}_{L}} {σ}_{{P}_{1} - {P}_{2}} , {b}_{9} = \frac{{U}_{R}}{{R}_{10}} {σ}_{{P}_{9} - {P}_{10}}$$

$${b}_{10} = - {U}_{R} ( \frac{{σ}_{{P}_{9} - {P}_{10}}}{{R}_{10}} + \frac{{σ}_{{P}_{10} - {P}_{11}}}{{\tilde{R}}_{R}} )$$

$${b}_{11} = \frac{{U}_{R}}{{\tilde{R}}_{R}} {σ}_{{P}_{10} - {P}_{11}} , {b}_{13} = \frac{{U}_{L}}{{R}_{13 b}} {σ}_{{P}_{13} - {P}_{1} .}$$

The closed-loop model presented in the present application reproduces the predominant features of the physiology of the human cardiovascular system that give rise to the BCG signal, and represents a novel theoretical interpretation of the BCG signal based on a physically-based (biophysical), mathematical closed-loop model of the cardiovascular system. Validation has been provided with comparison against actual measurements, matching not just qualitatively but also quantitatively.

Thus, as described herein, the present application discloses use of a mathematical model (i to simulate the motion of the center of mass of the body based on the physiology of the cardiovascular system, (ii) to interpret the BCGs acquired with different sensing modalities, and (iii) detect cardiovascular abnormalities for early diagnosis of pathologies. A theoretical BCG root signal was obtained by modeling the cardiovascular system as a closed-loop network of resistors, capacitors, inductors, voltage sources and switches arranged into four main interconnected compartments corresponding to the heart and the systemic, pulmonary and cerebral circulations (see FIG. 1). The mathematical modeling was used to predict how the root BCG signal would appear when measured by an accelerometer placed, for example, on a suspended bed and/or by a hydraulic sensor placed under a mattress such as an air mattress (see FIG. 2). The mechanistic descriptions described herein include characterization of both root and measured BCG signals in healthy and diseased subjects. In order to correctly interpret BCG signals for the early detection of CVD, it is necessary to characterize (i) the cardiovascular mechanisms (e.g., ventricular pumps, vascular resistance and compliance) giving rise to the root BCG signal in healthy conditions, (ii) the alterations induced on the BCG signal by specific abnormal cardiovascular conditions (e.g., heart failure, hypertensive diseases), and (iii) the relationship between root BCG signals and signals measured by a particular device. As described above, the cardiovascular model 10 was simulated via software to simulate aspects such as: (i) the cardiovascular mechanisms (specifically ventricular pumps, valvular function, vascular resistance and compliance) giving rise to a BCG in healthy conditions, (ii) the alterations induced on the BCG signal by specific abnormal cardiovascular conditions (heart failure, valvular diseases, hypertensive diseases), and (iii) the relationship between root BCG signals and signals measured by a particular device (e.g., an accelerometer, hydraulic bed sensor, etc.). By way of the improved closed-loop model 10 of the present invention, it is possible to (i) model and validate other sensing modalities, such as an accelerometer on a recliner chair and/or a hydraulic sensor placed under different types of mattresses, and (ii) characterize pathological changes in root and measured BCG signals.

Accordingly, the present invention provides for (i) a novel, complete cause-and-effect model to interpret BCG measurements from signals generated by the body motion to signals produced by different sensing modalities; (ii) a platform for innovative passive, noncontact monitoring of the cardiovascular status of patients at risk, without interfering with their daily life; (iii) novel opportunities for early detection of pathological cardiovascular conditions; and (iv) a modeling approach that could be applied to other studies of the cardiovascular system (discussed in the additional embodiment below). This represents an advancement in the current knowledge of BCG physiology in health and disease, and will help detect pathological abnormalities in a given patient, thereby having a significant impact on both theoretical and applied aspects of cardiovascular research.

With respect to applying the model 10 to improve sensor technology, the following embodiment is provided. To obtain real-world force measurements, a sensing device such as an accelerometer or other sensor may be used. For example, a hydraulic sensor as disclosed in US Patent Application Publication 2013/0197375 (incorporated by reference herein in its entirety) is such a sensor envisioned for obtaining real-world measurements. With respect to an accelerometer, use of the piezoelectric effect (e.g., via microscopic crystal structures that get stressed by accelerative forces, which causes a voltage to be generated), or the sensing of changes in capacitance are known sensing methods of an accelerometer. For accelerometer capacitance sensing techniques, a MEMS arrangement is used which provides for adjacent microstructures positioned next to each other, one being fixed, another being movable. These structures have a certain capacitance between them, such that if an accelerative force moves the movable structure, the capacitance will change, with such change being able to be measured. MEMS accelerometers with a noise density below 150 μm/s²/√Hz are commercially available, and envisioned for usage herewith. Adding additional circuitry to convert such measured capacitance by the accelerometer to a voltage signal then results in a traditional accelerometer sensor that outputs values representative of motion.

In the present application, the model 10 can be programmed into an integrated circuit (IC) that is integrally packaged with an accelerometer or other similar sensing component as described above, so long as the sensing component has the capability to sense real-world forces associated with cardiovascular events/functions/mechanisms. However, the model 10 can alternatively be programmed into an IC or computer system that is at a remote location from the sensing location (e.g., in the context of FIG. 2, remote from the bed 20). Thus, both localized (e.g., IC with model 10 programmed thereon being integrally packaged with the accelerometer) and remote (e.g., the model 10 is programmed into a computer in the form of a cloud-based computer processing system) proximities are envisioned with respect to the location of the IC or computer programmed with the model 10. The IC receives and analyzes/interprets the data output by the accelerometer (e.g., the data from the accelerometer being a representation of the real-world measurements sensed by the accelerometer). Such analyzation/interpretation in the programmed IC comprises comparing the data representative of the measured forces from the accelerometer to the parameter values associated with model 10 to determine a degree of correlation between the parameter values of the model and the data corresponding to the measured force values. A high degree of correlation (e.g., equal to or above a pre-determined threshold) indicates a match that would, for example, be deemed representative of the presence or occurrence of particular CVD or other cardiovascular function that the model 10 was tuned to detect. Thus, an overall sensor device or assembly that comprises a sensing element such as an accelerometer in combination with an IC programmed with model 10, would be able to detect whatever particular cardiovascular mechanism the model 10 was designed to approximate or predict, thereby allowing for the improved detection and/or monitoring of the cardiovascular characteristics (aka cardiovascular status) described herein.

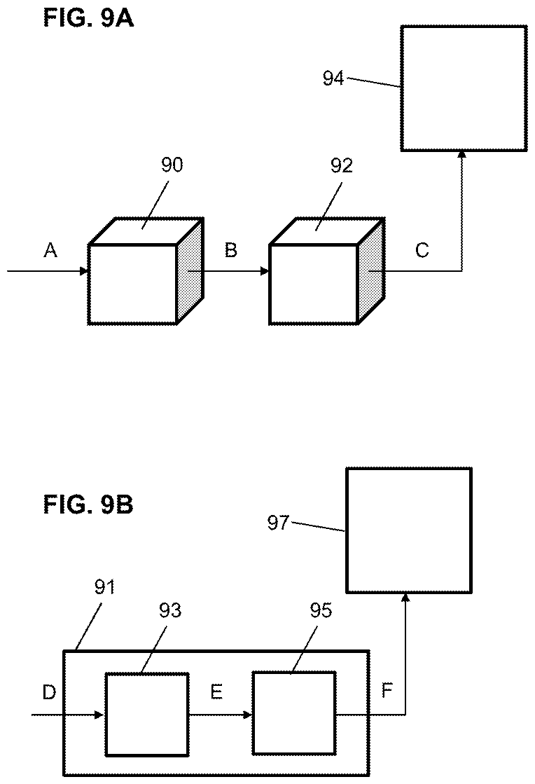

FIG. 9A represents a portrayal of how such a sensor system described above would operate. Real-world cardiovascular forces, represented by arrow A, are sensed by the real-world force detection sensor 90 (e.g., an accelerometer). The accelerometer 90 outputs signals (aka data) representing such forces at arrow B. The programmed IC 92 receives the output signals B from the accelerometer 90, and compares the signals B to the model parameters stored in the IC 92. Of course the IC 92 may comprise circuitry such as internal logic, registers and/or other memory elements as is known in the art, necessary for providing processing, storage and computing functions. External memories may also be utilized. The IC 92 may be part of a cloud-based computing system or other remote location computing system. If there exists a predetermined amount of matching between the data representing the measured forces and the parameters of the model, the output C of the IC 92 is then sent to a computing terminal 94 for further interpretation and/or processing and/or displaying. Such matching is indicative of the particular cardiovascular mechanism that the model 10 is formulated to approximate, such as a BCG in this case. For example, comparison of a measured BCG vs a BCG as simulated by model 10 would reveal the amount of deviation between the measured BCG and the BCG as simulated by the model 10. Analysis of the BCG signals can include investigation of various cardiovascular markers associated with BCG signals in order to detect cardiovasculars disease or other cardiovascular abnormalities. A determination can then be made as to whether or not there exists minimal deviation such that the simulated BCG accurately approximates the measured BCG. Computer terminal 94 can be any type of computer as is known, including a desktop, laptop, mobile phone, tablet, and so on and so forth, and may be equipped with an operating system and additional software for interpreting the output C of the IC 92. IC 92 itself may be programmed with additional software or extensions in association with the model 10, to provide additional analytical and/or processing functionality. Such additional software may provide the ability to parse BCG signals for known biomarkers and the like. The computer terminal 94 may of course include a visual display (not shown) for the conveying and displaying of information, and may have data connectivity (aka input/output) ports and wireless communication hardware as is known in the art. Elements 90 and 92 may likewise be individually capable of wireless communication by such wireless protocols including WiFi, Bluetooth, Z-Wave, Zigbee, and so on and so forth.

FIG. 9B shows an embodiment where a sensing element and IC are in a common component package 91. Similar to FIG. 9A, a sensing element 93 senses real-world forces represented by arrow D, outputs signals (aka data) representative of such forces at arrow E to IC 95 that has been programmed according to model 10. IC 95 then outputs data at arrow F to a computer terminal 97. The sensing element 93 can be any sensing element capable of detecting cardiovascular forces as described above, including but not limited to an accelerometer, a hydraulic sensor, and the like. The IC 95 can be a custom IC or an off-the-shelf IC. Because the overall package 91 can be small in size, the package 91 can be installed in or on objects such as beds, chairs, or even used in wearable technology applications, thus being able to detect and/or monitor cardiovascular functions in a continuous, non-invasive manner. The elements 93. 95 and 97 may have the same wireless communication features as described above for elements 90, 92 and 94 in FIG. 9A.

For example, such a system as in FIG. 9B can be used in a clinical setting such as a doctor's office or in an in-home setting. The physically-based BCG model 10 described herein can be integrated with techniques for monitoring patients (e.g., older adults) non-invasively in their home. An in-home sensor system incorporating the model 10 is capable of automatically generating health messages that indicate early signs of health change, thereby allowing for very early treatment, which has been shown to produce better health outcomes. While it is known to track parameters such as in-home gait patterns, overall activity level, heart rate and respiration rate, blood pressure, and sleep patterns, the novel model 10 and sensor application disclosed herein aids in the tracking of a person's cardiovascular health noninvasively by examining the morphology of the BCG signal. Using model 10 of the BCG sensing system, an output signal can be translated to the standard base signal for study against the simulated model output. Such study may be performed by a trained practitioner or by software. That is, the closed-loop model 10 is able to be used to simulate pathological conditions affecting the cardiovascular system, such as hypertension or congestive heart failure, and the results are compared to measured BCG signals. This represents significant potential in better monitoring of cardiovascular health, particularly important for aging populations.

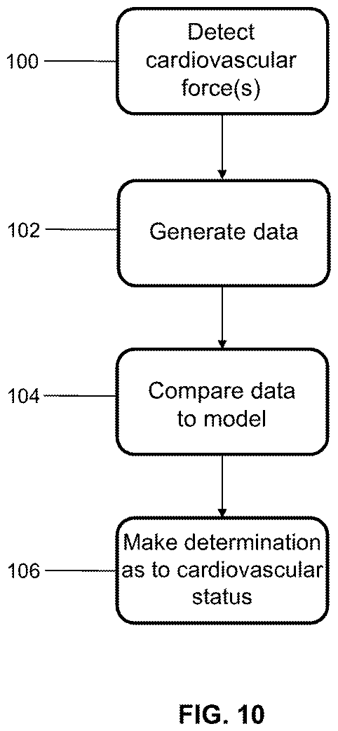

FIG. 10 illustrates a sample process flow for a method of acquisition of force data and comparison of such data to model 10. At step 100, cardiovascular forces are sensed by a sensing element as described in connection with FIGS. 9A and 9B. At step 102, the sensing element generates data representing such sensed forces. Such data may be individualized data points (e.g., each axis as described above for accelerometer 24a) or data extracted from a BCG signal that is generated from such individual data points. At step 104, the force data is compared to the parameters of model 10. At step 106, a determination is made as to whether the force data is representative of a particular cardiovascular function based on overlapping/matching data points between the force data and the model parameters. This can include, for example, comparing parameters as in Table III to a BCG produced from forces measured by the accelerometer in FIGS. 9A/9B. Thus, the sensing methodology technique described herein is capable of sensing real-world cardiovascular forces and determining if the forces are representative of known cardiovascular forces/functions/mechanisms/conditions as modeled by the parameters of model 10, representing an improvement over conventional cardiovascular sensing techniques and technology. This allows for development of particularized sensors that are tuned to detect a particular cardiovascular disease, and generate health alerts accordingly.

Accordingly, the BCG model is envisioned for use in clinical interpretation and for tracking cardiovascular health noninvasively. This includes model development for different BCG sensing systems, coupled with the BCG model herein. The combined BCG and sensor model is able to be applied for specific populations, for example, a male BCG model and a female BCG model that reflect the different physiology of the male and female cardiovascular systems, and in general personalization of the BCG model based on an individual's physiology.

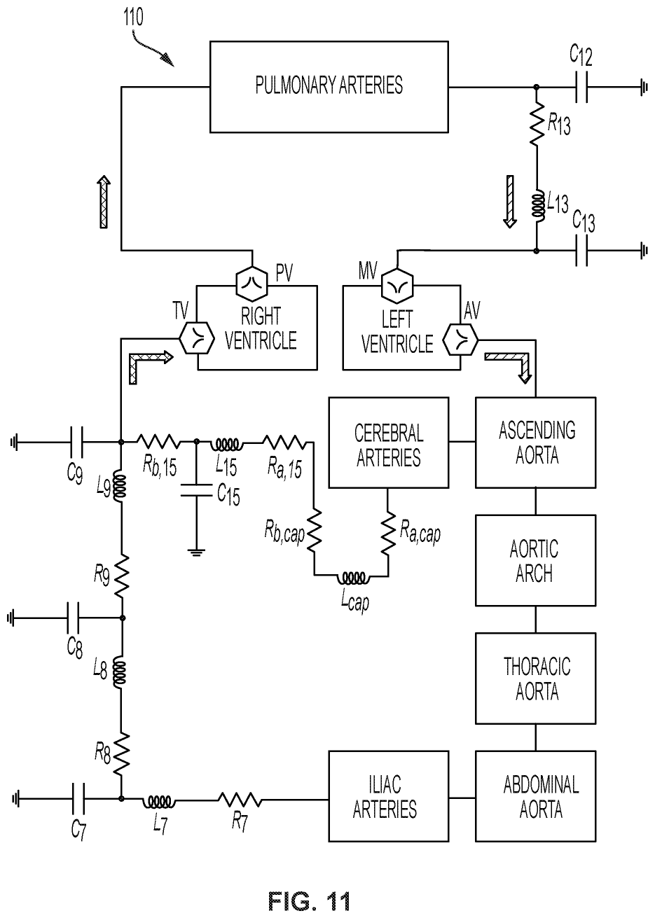

In another embodiment, the novel physically-based, circuit component modelling technique described herein is used in conjunction with modelling and assessment of VAC. FIG. 11 illustrates a schematic representation of a closed-loop model 110 utilized to simulate blood flow through the cardiovascular system, and shows representations of the tricuspid valve (TV), pulmonary valve (PV), mitral valve (MV) and aortic valve (AV). Like model 10, the closed-loop model 110 leverages the analogy between electric systems and hydraulic networks, with electric potentials, electric charges, and electric currents corresponding to fluid pressures, fluid volumes and volumetric flow rates, respectively. As such, only those features of the model 110 that were utilized for VAC analysis are described, acknowledging that remaining aspects of the model 110 can be found in model 10.

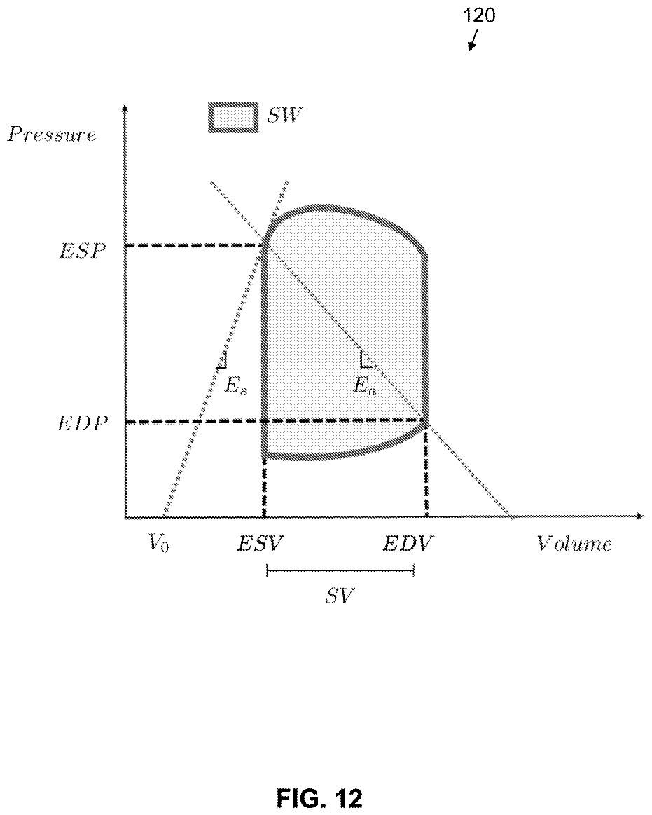

FIG. 12 illustrates a conventional pressure-volume curve 120 characterizing the efficiency of the left ventricle. In FIG. 12, ESV and EDV denote the end-systolic and end-diastolic volumes, ESP and EDP denote the end-systolic and end-diastolic pressures, V₀ denote the volume at zero pressure, and the area under the curve provides the stroke volume (SW). The slope of the EDV−ESP line provides a lumped parameter, known as arterial elastance (Eₐ), which represents the arterial load. The slope of the V₀−ESP line provides the end-systolic ventricular elastance (Eₛ), which is considered a reliable clinical index of myocardial contractility. Finally, as discussed above, VAC is calculated as the ratio of the effective arterial elastance (Eₐ) to the end-systolic elastance (Eₛ), namely VAC=Eₐ/Eₛ. Overall, this ratio provides a well-established framework to characterize the ventriculoarterial interaction and it has been utilized in several human and animal studies.

However, for reasons discussed above, estimation of VAC via the pressure-volume curve suffers from several drawbacks. Application of the closed-loop model 110 of the present application to VAC therefore represents an improvement over the art with respect to assessment of VAC. That is, the closed-loop model 110 provides for a non-invasive method for VAC assessment based on ECG, SCG and physically-based modeling.

Ventricular contractions trigger vibrations of the heart walls, valves, and blood, which propagate to the surface of the chest, generating an SCG signal. The SCG signal can be captured, for example, by way of an accelerometer (such as those described above) placed on the sternum of a subject (for example, in the setup shown in FIG. 2, a chest strap (not shown) including an accelerometer may be strapped around the chest of the subject 26 to measure forces and output an SCG signal)). While there are known algorithms for processing SCG signals, these algorithms only give the timing of the opening and closing of heart valves (namely, Mitral valve Opening (MO) and Closure (MC), isovolumetric contraction (IV), aortic valve opening (AO) and closure (AC)), and thus lack predictive ability. In order to arrive at a method with predictive ability of telling how these timings will change as VAC changes, a physically-based model of cardiovascular physiology is necessary. Thus, the closed-loop model 110 described herein has been used to develop a novel technique to assess VAC based on the analysis of the heart valve timings captured via SCG signals.

The individualized cardiovascular model 110 is used to predict the characteristic SCG signatures associated with changes in the elastance of the left ventricle or the main arteries. By embedding the physiological model 110 into the workflow, a sharper detection of valve timings is obtained, which improves the ability to detect changes in valve timings. The cardiovascular model 110 can be used as a virtual laboratory to predict how clinically relevant conditions, such as changes in ventricular and/or arterial elastances, are going to affect valve timings.

Synchronous recordings of ECG and SCG signals were acquired on (human) test subjects. The acquired signals were processed and analyzed. The simultaneous recordings of the ECG and SCG signals were used to adjust the input parameters of the closed-loop cardiovascular model. For example, beat-to-beat time intervals between consecutive ECG R-peaks were used as new input parameters for the closed-loop model 110 of the cardiovascular system. In doing so, individualization of the model 110 was achievable. Such individualization makes it possible to assess VAC based on valve timing. Post-processing (e.g., via Matlab) was used to obtain and analyze closing times for the mitral and aortic valves predicted via the individualized physiological model. Changes in the VAC state due to ventricular contractility or afterload were associated with non-invasively tracked heart valve timings. SCG interpretation based on ECG and cardiovascular modeling provided a sharp detection of valve timings, thereby allowing for assessment of VAC based on SCG.

The above was accomplished by testing of (human) test subjects, which included having the test subjects lay still on a bed for a predetermined amount of time (e.g., five minutes) while ECG and SCG signals were recorded simultaneously. For example, ECG signals were measured with a 3-lead configuration, and SCG signals were measured with a sensor (e.g., a Kionix KXR94-2283 accelerometer, with 1000 mV/g sensitivity) placed on the subjects' sternum. The ECG and SCG signals were collected simultaneously using a data acquisition system (e.g., an AD Instrument PowerLab 16/35 data acquisition system).

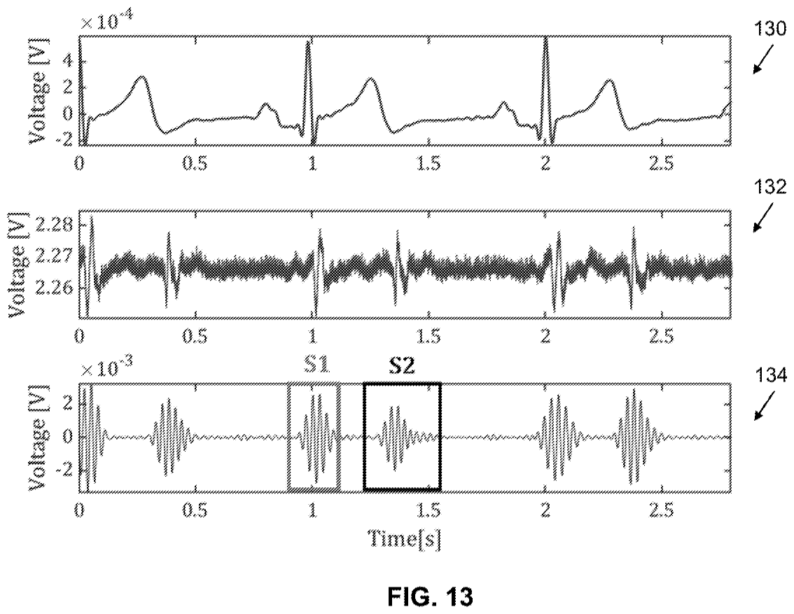

FIG. 13 shows plots of the analysis of the measurements. In FIG. 13, the top plot 130 represents an ECG signal, the center plot 132 represents a synchronous SCG signal, and the bottom plot 134 represents heart sounds generated by bandpass filtering of the SCG signal. R-peaks in the ECG signal were detected using the standard Pan-Tompkins algorithm, and used as a reference to detect heart sounds from the SCG signal. The SCG signal was processed via a bandpass filter (e.g., with a 20-250 Hz cut-off frequency). With respect to the bottom plot 134 in FIG. 13, it is known that the first heart sound (S1) is due to closure of the mitral and tricuspid valves at the start of systole, whereas the second heart sound (S2) is due to closure of the aortic and pulmonic sound due to the closure of the aortic and pulmonic valves, marking the end of the systole.

As described above, the novel closed-loop physiological model 110 for cardiovascular physiology can produce arterial pressure waveforms and ventricular functions that are in qualitative agreement with measured waveforms and functions, and thus represents a significant advancement in the field over conventional open-loop models. As the full details of the model 10 were discussed above, only those features of the model 110 utilized in the VAC embodiment are further discussed.

With further reference to FIG. 11, the circulation of blood through the cardiovascular system is driven by pumping action of the ventricles, which are modeled as voltage generators and variable capacitors connected in series.

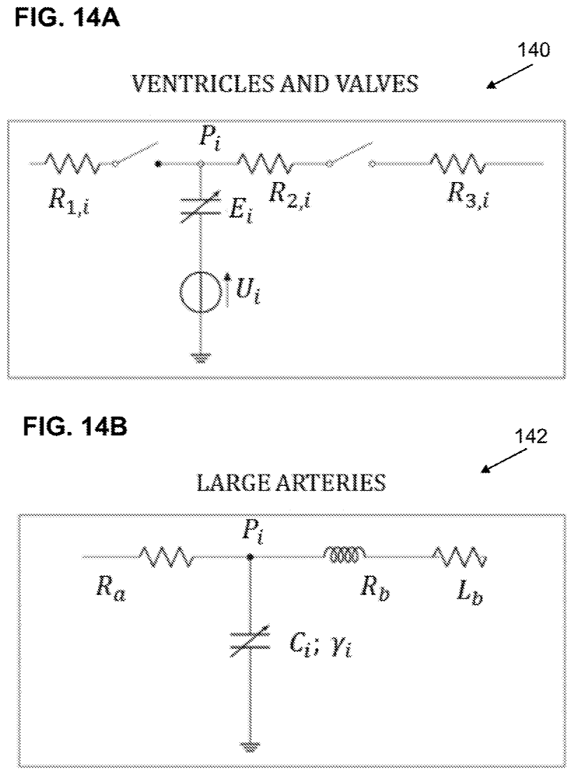

FIG. 14A shows circuit representations 140 of the ventricles and valves, and FIG. 14B shows circuit representations 142 of the large arteries. In FIG. 14A, the voltage generators, characterized by Uᵢ=Uᵢ(t) with i=L, R, describe the electrochemical activation of the ventricles, whereas the variable capacitors model the ventricular biomechanical properties by way of the time-varying elastance Eᵢ=Eᵢ(t).

Denoting by Pᵢ and Vᵢ, with i=L, R, pressure and volume of the left and right ventricles, respectively, results in Pᵢ=EᵢVᵢ with Equations (8) and (9) as follows:

E_{L}(t)=ELD+ELSa_{L}(t) (8)

E_{R}(t)=ERD+ERSa_{R}(t) (9)

where ELD, ELS, ERD and ERS are given constants characterizing the systolic and diastolic elastances of the left and right ventricles, whereas a_{L}(t) and a_{R}(t) are the activation functions characterizing the timing of the ventricle contractions. A known activation function is described mathematically in Equation (10) as:

$${a}_{i} ( {t}_{m} ) = \{ \begin{matrix}\frac{tanh ( 2 π ( {t}_{m} - {T}_{a} ) ) - tanh ( 2 π ( {t}_{m} - {T}_{b} ) )}{2} \\ 0 \end{matrix} {t}_{m} < {T}_{s} \qquad ( 10 )$$

Otherwise for i=L, R, where tₘ is defined as tₘ=mod(t, T_{c}), T_{c} being the length of the cardiac cycle and Tₛ being the length of the systolic part of the cardiac cycle. The time constants Tₐ and T_{b} can be characterized via electrocardiography. Specifically, Tₐ corresponds to the T wave peak time and T_{b} corresponds to the T wave offset time with respect to the R wave peak in the ECG. As shown in FIG. 14A, the heart valves are modeled as ideal switches which close as soon as the pressure gradient across them is favorable.

FIG. 14B shows circuit representation 142 of the large arteries, namely ascending aorta, aortic arch, thoracic aorta, abdominal aorta, iliac arteries and cerebral arteries, sharing the same mathematical description, which include two resistors, one inductor and one variable capacitor representing hydraulic resistance, inertial effects and wall compliance, respectively.

The constitutive laws defining these elements are:

$$( resistor ) Δ P = RQ \\ ( inductor ) Δ P = L \frac{dQ}{dt}$$

$$( variable capacitor ) Δ P = \frac{V}{C} + γ \frac{dV}{dt}$$

where ΔP is the pressure difference across the element, Q is the volumetric flow rate, V is the fluid volume, R is the hydraulic resistance, L is the inductance, and C and γ are positive constants representing the elastic and viscoelastic properties of the vessels wall. The parameter values characterizing the main arteries were computed using the following relationships, as were discussed earlier:

$$R = \frac{8 π l η}{{S}^{2}} , L = \frac{{ρ}_{b} l}{S} , C = \frac{3 {lS ( a + 1 )}^{2}}{E ( 2 a + 1 )} , γ = \frac{δ}{C}$$

where l is the vessel length, a=r/h is the ratio between the vessel radius r and the wall thickness h, η is the blood viscosity, S=πr² is the vessel cross-sectional area, ρ_{b} is the blood density, E is the Young modulus and δ is a viscoelastic parameter, as discussed above.

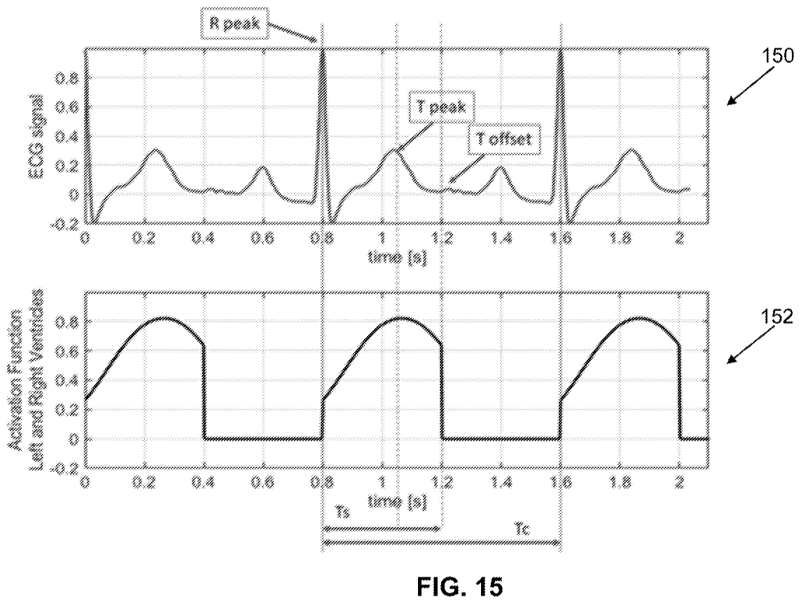

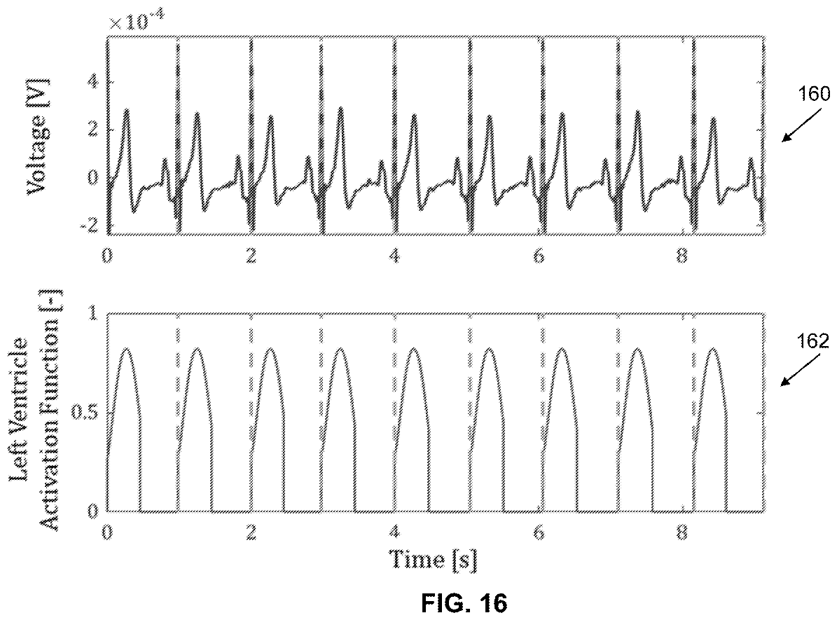

With respect to individualization, the shape of the activation functions aᵢ(t), with i=L, R, can be tightly related to some specific events in the ECG. Specifically, as shown in FIG. 15, the contraction of the ventricle begins at the R-peak, and the end of the systolic phase corresponds to the T wave offset time, with the peak of the activation function occurring at the T-peak. The top plot 150 of FIG. 15 represents a sample ECG signal, with important key points for cardiac electric timing. The bottom plot 152 of FIG. 15 represents a graph of the activation function given in Equation (10) for modelling the pumping functions of the ventricles (where T_{c}=0.8 s and Tₛ=0.4 s). Although the model requires several input parameters discussed in the prior embodiment, the focus was on adapting the length of the cardiac cycle T_{c} to that dictated by the ECG signal. As a result, as shown in plots 160 and 162 of FIG. 16, model individualization was achieved by adapting the above-noted activation function to the ECG R-peak, and the timing of the present model dynamically adapts to the real heart rate of the subjects.

The following five steps were applied to generate individualized outputs from the physiological closed-loop model: 1) filter ECG signals for high-frequency noise and low frequency respiratory variations; 2) use the standard Pan-Tompkins algorithms to detect the ECG R-peaks; 3) define the length of each cardiac cycle in the model according to the RR-intervals extracted from ECG; 4) for each RR-interval, let the closed-loop model run and generate 20 cardiac cycles; and 5) consider the last cycle as a representative solution.

Post-processing can be performed with any suitable software, including but not limited to Matlab and the like. For example, post-processing of the model in Matlab can be performed to locate the aortic and mitral closing times. The aortic closing time is defined as the instant corresponding to a zero flux through the aortic valve, while the mitral closing time is defined as the instant corresponding to a zero flux through the mitral valve.

The closed-loop physiological model 110 was evaluated on three aspects, including i) capability of the closed-loop model to predict changes in the pressure-volume curve of the left ventricle due to changes in ventricular and arterial elastances, (ii) reliability of the individualization techniques with respect to detecting valve timing in (human) test subjects, and (iii) feasibility of utilizing valve timing to estimate the VAC.

The closed-loop model 110 was able to predict changes in the pressure-volume curve of the left ventricle due to changes in ventricular and arterial elastances. That is, the physiological closed-loop model can predict a functional change in the shape of the pressure-volume curve of the left ventricle as a result of an increased ventricular contractility and a reduced afterload.

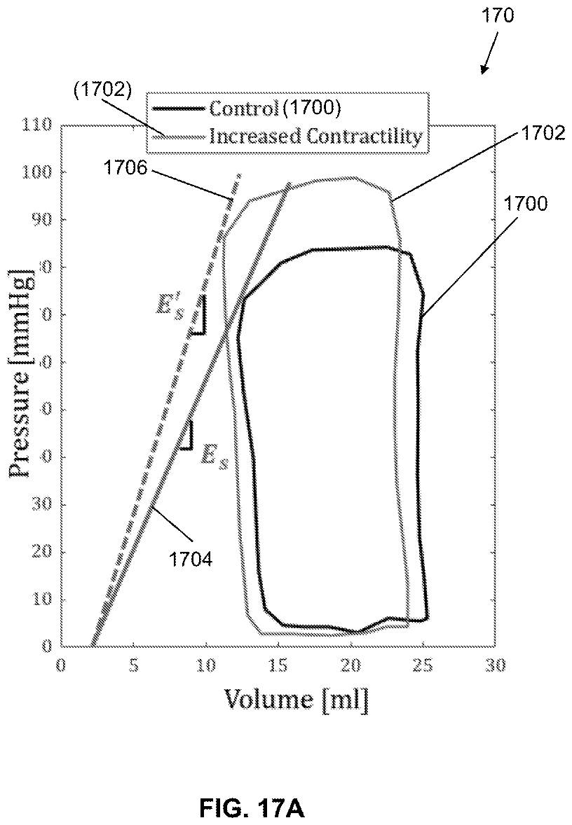

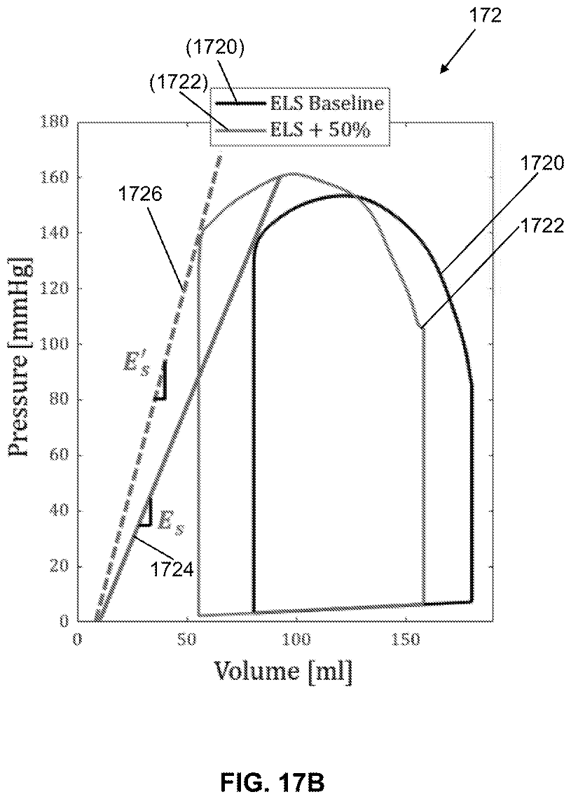

For example, in FIG. 17A, known pressure-volume curves as measured from a canine left ventricle at control state and after epinephrine injection are shown. That is, shown in the figure is a plot 170 of a configuration characterized by a control contractile state 1700, and an enhanced contractile state 1702 resulting from the injection of epinephrine. The main detectable changes are that the end systolic elastance E_{S} increases according to the slope of the Eₛ line 1704, the curve shifts to the left towards smaller volumes, and the curve stretches upward reaching higher pressures. An E′ₛ line 1706 is also shown.

In FIG. 17B, plot 172 shows pressure-volume curves simulated via the closed-loop model for ELS (see Equation (8)) at baseline (1720) and at a 50% increase over baseline (1722). The slope of the Eₛ (1724) and E′ₛ (1726) lines represent the end-systolic elastance Eₛ. The closed-loop model estimates the pressure-volume curves shown in FIG. 17B, obtained by varying the parameter ELS introduced in Equation (8). The simulated control state represented by the solid curve was obtained with ELS at a baseline value of 1.375 mmHg cm⁻³ s⁻¹, whereas the state of (increased) contractility represented by the dashed curve was obtained by increasing ELS of 50% over baseline. The simulated results show an increase in Eₛ, a shift towards lower volumes and a stretch to higher pressures, as expected from measured physiological results (e.g., FIG. 17A).

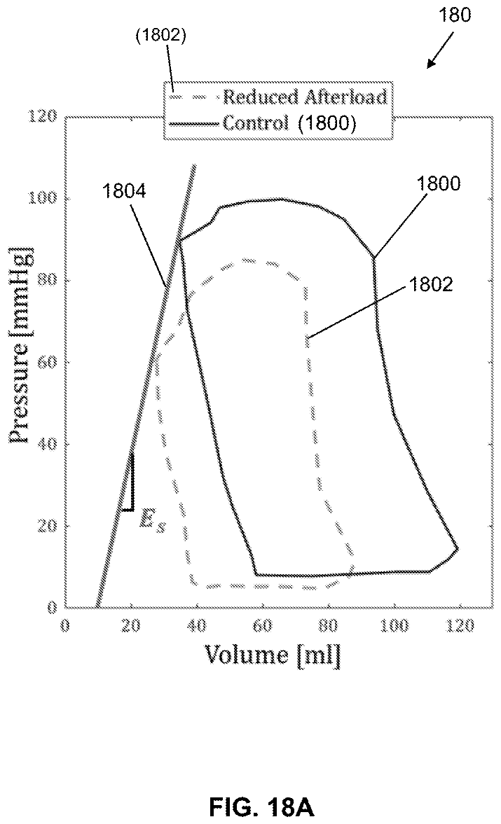

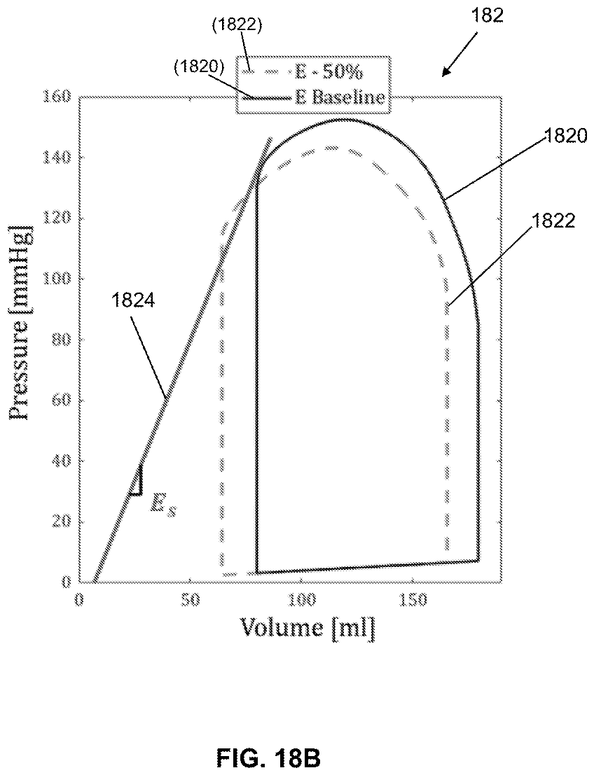

Other aspects such as a reduced afterload were also evaluated. In clinical settings, reduced afterload can achieved by administration of nitroglycerin in test subjects. FIG. 18A shows pressure-volume curves known in the art, measured in the left ventricle of a patient at control state (solid curve 1800) and after administration of nitroglycerin (dashed curve 1802), as shown in plot 180. In the case of reduced afterload, it was observed that (i) the end systolic elastance Eₛ does not change significantly (as evidenced by the slope of the Eₛ line 1804), (ii) the curve shifts to the left towards smaller volumes, and (iii) the curve shrinks downward reaching lower pressures.

Such reduced afterload conditions were simulated using the closed-loop model 110. FIG. 18B illustrates pressure-volume curves simulated via the closed-loop model for E (e.g., as shown in the above-noted relationship equation for calculating C) at baseline (solid curve 1820) and at 50% below baseline (dashed curve 1822), as shown in plot 182. The slope of the Eₛ line 1824 represents the end systolic elastance Eₛ. Reduced afterload conditions were simulated using the closed-loop model by reducing arterial Young Modulus E, thereby reducing the capacitance in large arteries and thus the load against which the ventricle must eject. For example, the simulation used a 50% reduction with respect to a baseline value of 4·10⁶ dyne cm⁻². As shown, the simulation results display the same characteristics observed in the measured results (e.g., FIG. 18A), thereby demonstrating the capability of the closed-loop model to predict changes in the pressure-volume curve of the left ventricle associated with changes in the physiological conditions. In particular, reduced ventricular contractility and increased afterload can be simulated by modifying the numerical values of ELS and E, as shown in Table VI:

TABLE VI
PATHOLOGICAL CONDITIONS AND
CORRESPONDING SIMULATED SCENARIOS
WITH THE CLOSED-LOOP MODEL.

| PATHOLOGICAL CONDITION | MODEL SIMULATION |
|---|---|
| Reduced ventricular contractility | Reduced ELS |
| Increased atterload | Increased E |

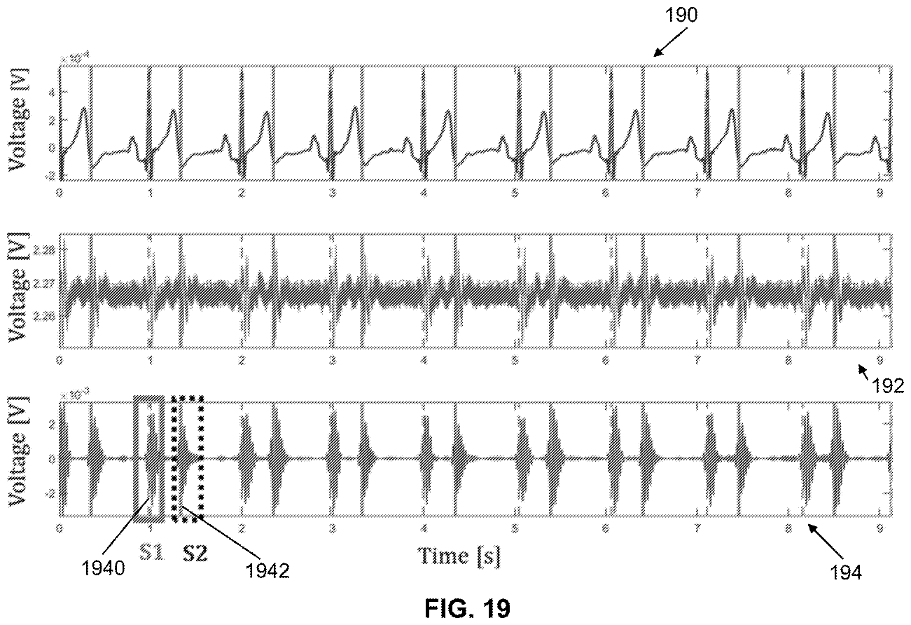

Individualization of the model-based procedure for valve timing was carried out. FIG. 19 shows a sample segment of a simultaneously recorded ECG 190 and SCG 192 for a test subject, along with a corresponding filtered heart sound 194 acquired from the SCG 192. Superimposed thereon is the closed-loop model based estimation of aortic and mitral valve closures, obtained from simulated flows through the valves after adapting the heart beat in the model to that given by the ECG, thus providing a beat-to-beat individualized estimate of the valve closures. As shown, there is substantial alignment of the recorded heart valve sound and the valve timing predicted by the closed-loop model. For example, the model-based estimates of the mitral valve closure (dashed line 1940) occurs at the beginning of the first heart sound (S1) at the beginning of the systolic period. Similarly, the model-predicted aortic valve closure (solid line 1942) falls at the onset of the second heart sound (S2) at the beginning of the diastolic period. Such agreement between the measured and estimated graphs was sustained throughout all nine beats of the test subject, thereby demonstrating the qualitative predictive capability of the closed-loop model 110 to estimate valve closing times on an individualized basis (e.g., specific to the patient), a noted improvement over conventional techniques.

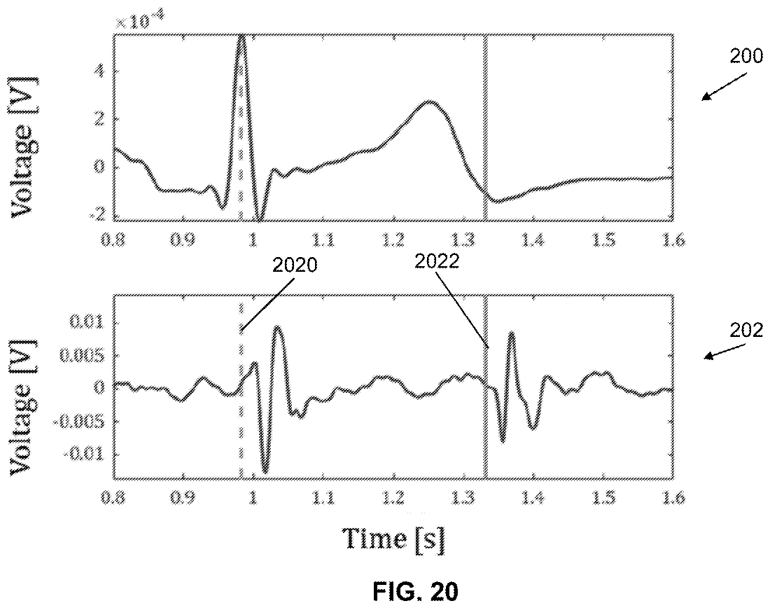

FIG. 20 shows simultaneous recordings of ECG 200 and SCG 202 signals collected during one heartbeat, and indicates that mitral valve closure (dashed line 2020) occurs in the proximity of the first peak of the SCG, right before the largest SCG peak. Additionally, closure of the aortic valve (solid line 2022) appears in a region between a peak and the following valley in the SCG, at the end of the ECG T-wave. These results comport with expected results from conventionally measured signals.

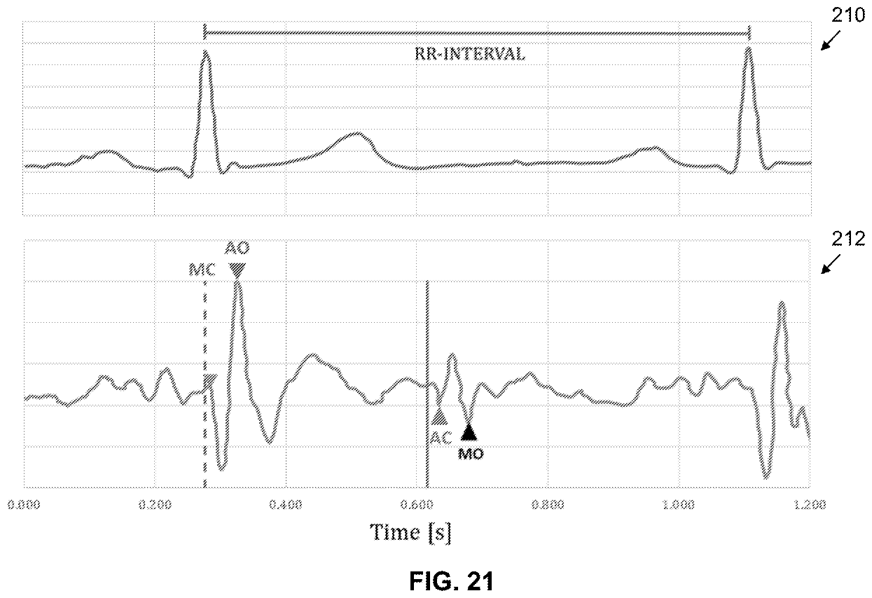

In FIG. 21, plots 210 and 212 show a comparison between valve timings estimated with the individualized closed-loop model of the present application and those obtained via conventional SCG analysis. That is, the comparison is between known valve timings from the SCG signal via the method based on envelope signal filtering and envelope calculation (represented by downward triangles), and those estimated via the present individualized model-based procedure (represented by vertical lines). In FIG. 21, in plot 212, MC represents mitral valve closure, AO represents aortic valve opening, AC represents aortic valve closure, and MO represents mitral valve opening. Specifically, by extracting the RR-interval associated with the sample heartbeat in plot 210 of FIG. 21, individualized estimation of the mitral and aortic valves is possible. The computed timings were superimposed on conventionally-measured timings, with SCG fiducial points (indicated by the downward triangles) in FIG. 21 being obtained by conventional signal filtering techniques. The plot 212 of FIG. 21 shows that valve closures estimated with the present individualized closed-loop model are consistent with conventionally measured valve timings.

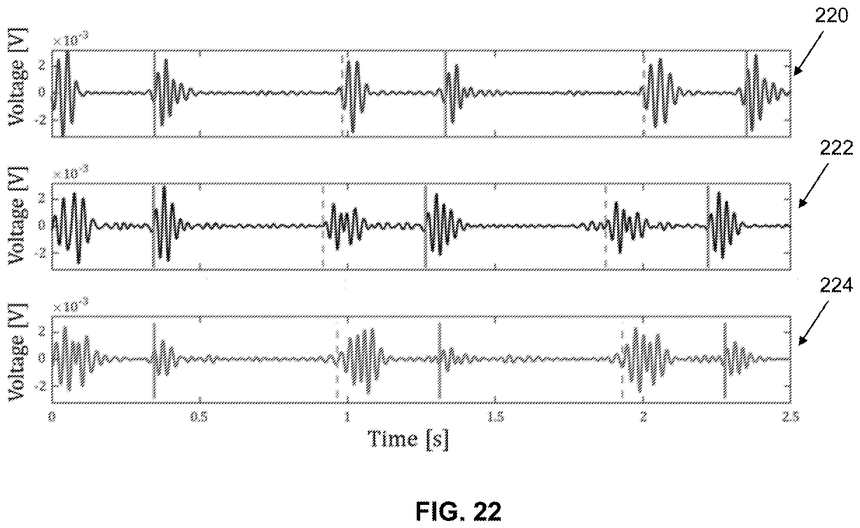

FIG. 22 shows filtered SCG signals for each of the three test subjects (test subject 1 shown at 220, test subject 2 shown at 222, and test subject 3 shown at 224) that were studied, along with valve timings predicted by the individualized closed-loop model of the present application (see vertical lines). Namely, filtered SCG signals for each of the three subjects that were studied are displayed along with the mitral valve closures (dashed vertical lines) and the aortic valve closures (solid vertical lines) predicted by the individualized model-based procedure In all subjects, the mitral valve closure occurs in the first valley of the S2 sounds. The results show that the method of individualizing the closed-loop model of the present application results in consistent estimates of the timing of the valve closures for each test subject, thereby confirming the predictive ability of the (individualized) closed-loop model disclosed herein.

Due to the above-noted manner in which the closed-loop model 110 of the present application is able to accurately predict and track conventional measurements and be individualized with patient-specific parameters, the closed-loop model can be used as the basis for improving sensor technology in the field of cardiovascular detection and/or monitoring. As shown by the examples herein, the physiological closed-loop model is capable of predicting changes in the pressure-volume curve of the left ventricle associated with VAC changes due to abnormal ventricular or arterial elastances, and estimating closing times of the mitral and aortic valve based on ECG and SCG measurements.

To confirm the viability of estimating VAC non-invasively via ECG and SCG measurements, three variables were created, including: LVET (left ventricular ejection time, defined as the interval between the closing times of aortic and mitral valves); Q_{MV} (maximum flow through the mitral valve); and Q_{AV} (maximum flow through the aortic valve). While only LVET can be obtained via processing of SCG signals, Q_{MV} and Q_{AV} are hemodynamic variables that can be estimated via the physiological closed-loop model. Model results indicate that changes in the VAC state due to ventricular contractility or afterload are both associated with variation in Q_{AV}, whereas only an increased afterload leads to a significant change in the LVET. The SCG interpretation based on ECG and cardiovascular modeling provides a sharp detection of valve timings that makes it possible to assess VAC based on SCG.

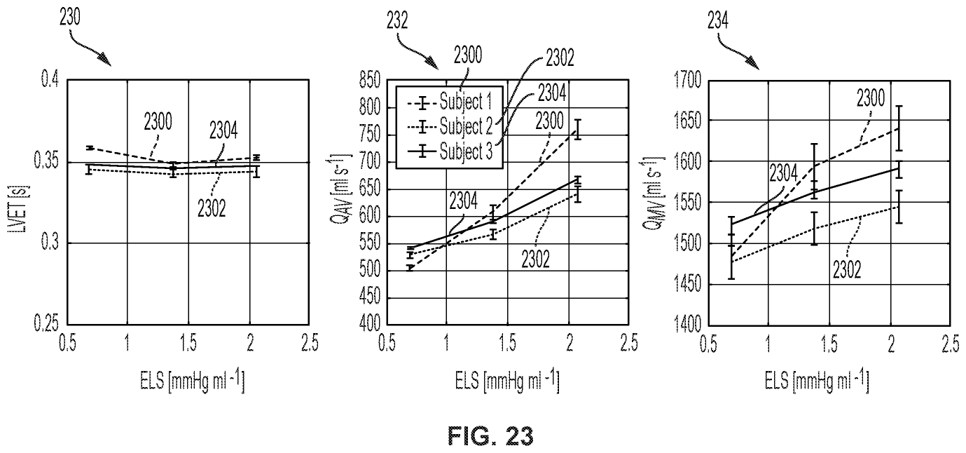

FIG. 23 shows predicted values of LVET (in plot 230), Q_{AV} (in plot 232) and Q_{MV} (in plot 234) for hypothetical increments and decrements of 50% in the value of ELS for each of the test subjects (subject 1 (2300), subject 2 (2302) and subject 3 (2304)). Specifically, the histograms were obtained by utilizing the RR-interval extracted from each subject and then performing the simulations of the closed-loop model with a modified ELS value. That is, shown is variation in the timing of the closure of the aortic valve (plot 230), the maximum flux through the aortic valve (plot 232) and the maximum flux through the mitral valve (plot 234) for three different values of the end systolic elastance ELS. ELS changes are indicative of altered ventricular contractility, as it is quite often observed in the case of severe shock. The graph reports the average values obtained with the simulations, along with the range of variations due to heart rate variability. Model results indicate that a change in VAC associated with an altered contractile state is mainly detected analyzing Q_{AV} (this trend was consistent in all the test subjects). Changes in ELS are expected to induce slight changes in Q_{MV} and only minimal variations in LVET.

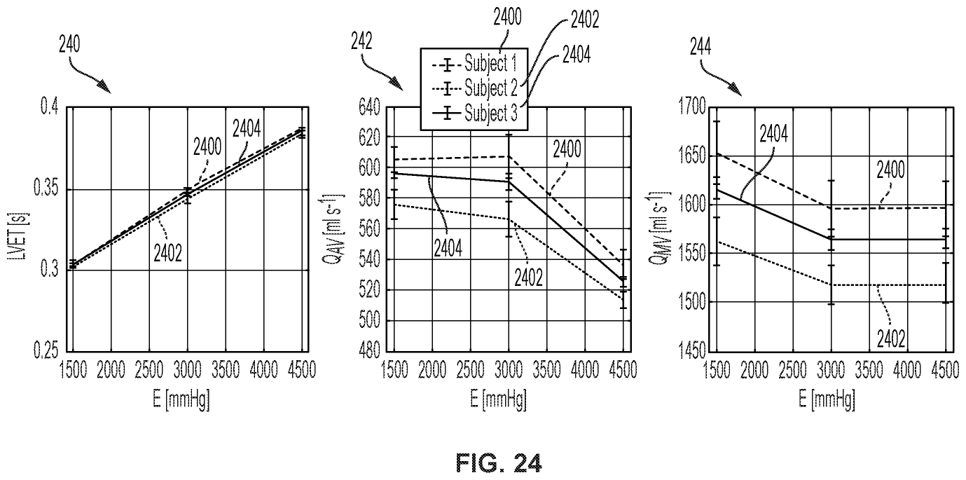

FIG. 24 shows the predicted values of LVET (in plot 240), Q_{AV} (in plot 242) and Q_{MV} (in plot 244) for hypothetical increments and decrements of 50% in the value of the arterial Young modulus E for each of the test subjects (subject 1 (2400), subject 2 (2402) and subject 3 (2404)). The left plot 240 of FIG. 24 shows variation in the timing LVET of the closure of the aortic valve. The middle plot 242 of FIG. 24 shows the maximum flux Q_{AV} through the aortic valve, and the right plot 244 in FIG. 24 shows the maximum flux through Q_{MV} the mitral valve for three different values of the Young Modulus E. As discussed, changes in E are associated with an abnormal afterload, as it is quite often encountered in intravascular obstructive shock conditions. Model results indicate that a change in VAC associated with an altered arterial elastance is mainly detected by analyzing LVET (whereas changes in E are expected to induce only moderate changes Q_{AV} and only minimal variations in Q_{MV}). The amount of change in LVET was consistent across all the test subjects. Overall, model results indicated that changes in the VAC state due to ventricular contractility or afterload are both associated with variation in Q_{AV}, whereas only an increased afterload leads to a significant change in the LVET, as summarized in Table VII:

TABLE VII
PARAMETERS ASSOCIATED WITH
ALTERATION OF THE VAC STATE

| Condition | LVET | QAV | QMV |
|---|---|---|---|
| Reduced ventricular contractility | ≈ | ⬇ | ↓ |
| Increased afterload | [symbol] | ⬇ | ↓ |

By combining ECG and cardiovascular modeling to find LVET from SCG, a much sharper diagnostic tool is derived. Interpretation of physiological measurements (ECG, SCG) via mathematical modeling has been shown herein to reveal useful information in a clinical setting. Here, it has been shown that an abnormal valve timing, which intrinsically embeds information about the interplay between the left ventricle and the arterial system, is more likely to be expected in a condition characterized by an abnormal afterload rather than an abnormal ventricular contractility. Additionally, changes in ventricular contractility are able to be observed in the maximum flux through the aortic valve. The model 110 can be integrated with sensor technology in the manner described above in connection with FIGS. 9A, 9B and 10, to take advantage of such benefits provided by the model. While test subjects in the above testing were human, similar testing could be performed on other living things such as animals (e.g., mice, dogs, etc.).

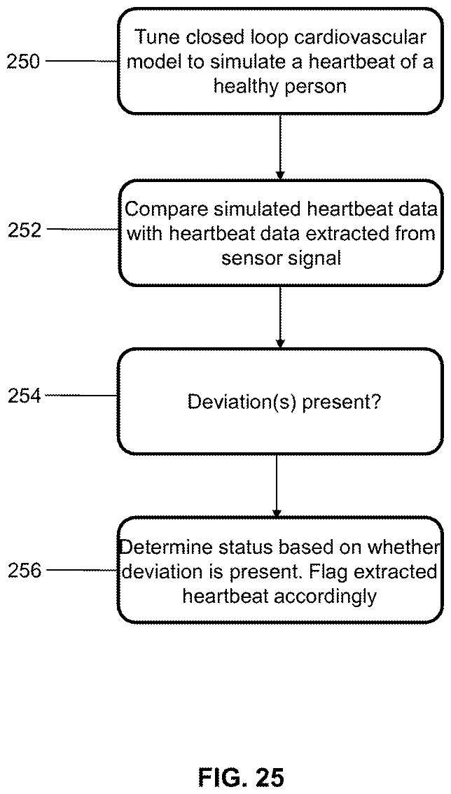

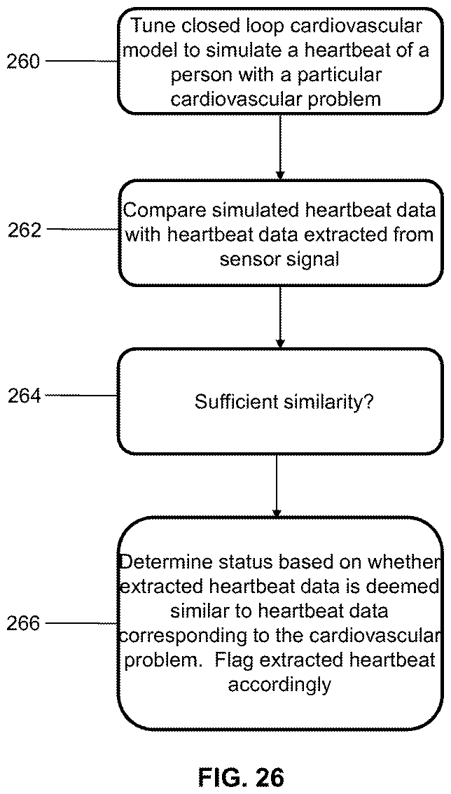

FIGS. 25 and 26 show process flows for example embodiments of implementing the closed loop cardiovascular models described above to improve the monitoring and detection of cardiac status.

In the example of FIG. 25, the closed-loop cardiovascular model is tuned to simulate a heartbeat of a healthy person (step 250). This can be accomplished by choosing parameter values (e.g., see Table III in FIG. 27), where the parameter values are chosen by a practitioner to cause the model simulate a known healthy heartbeat pattern. Such parameter values can be selected based on an analysis and determination of approximating circuit elements as physiological status as described above.

At step 252, circuitry compares the simulated heartbeat data produced by the model with heartbeat data that is extracted from the sensor signal produced by the sensor. Any of a number of sensor and signal processing circuit arrangements can be employed for extracting heartbeat data from a sensor signal. For example, US Patent Application Publication 2013/0197375, the entire disclosure of which is incorporated herein by reference, discloses various examples of sensor systems that can extract BCG-related heartbeat data from non-invasive pressure sensors such as hydraulic sensors that can be placed under a test subject's mattress while resting or sleeping. The comparison at step 252 can use correlation or other waveform matching techniques to assess the similarity between the simulated “healthy” heartbeat data and the extracted heartbeat data from the sensor.

At step 254, the circuitry determines, based on the comparison at step 252, whether there is a deviation between the simulated “healthy” heartbeat data and the extracted heartbeat data from the sensor. To support this determination, thresholds can be defined that establish tolerances around the simulated healthy heartbeat data to reduce the risk of false positives. In other examples, step 254 can search the extracted heartbeat data for specific deviations from the simulated “healthy” heartbeat data that are known to be markers for adverse cardiac conditions or pathologies, as discussed above.

At step 256, the circuitry can determine a status for the test subject based on whether any deviations were detected at step 254. The extracted heartbeat data may then be flagged accordingly with a status indicator (e.g., that may be automatically generated in response to a certain analytical result). The flagged heartbeat data can then be provided to a reviewer for further evaluation if desired. For example, if step 254 results in a determination that no deviations from the simulated “healthy” heartbeat were detected, then the extracted heartbeat data can be flagged with a “normal” or “healthy” indicator. Similarly, if step 254 results in a determination that one or more deviations from the simulated “healthy” heartbeat were detected, then the extracted heartbeat data can be flagged with a “alert” indicator or some other indicator of a specific condition if the system is able to map deviations with specific conditions. In this fashion, the process flow of FIG. 25 supports the ability to quickly and accurately detect potential cardiac conditions and flag those conditions for follow-up such as via alert notifications. Examples of sensor systems that support alert generations and which can be augmented with advanced detection capabilities as a result of the closed loop cardiovascular modeling discussed herein are described in U.S. Pat. No. 10,188,295, the entire disclosure of which is incorporated herein by reference.

In the example of FIG. 26, the closed-loop cardiovascular model is tuned to simulate a heartbeat of a person with a specific cardiac problem (step 260). This can be accomplished by choosing parameter values (e.g., see Table III in FIG. 27), where the parameter values are chosen by a practitioner to cause the model simulate the known heartbeat patterns of a person suffering from that cardiac problem.

Step 262 can be proceed in a fashion similar to that described above for step 252 of FIG. 25. The difference relative to FIG. 25 can be implemented at step 264. Whereas step 254 is searching for deviations between the simulated “healthy” heartbeat and the extracted heartbeat data of the test subject, step 264 is looking for similarities between the simulated “unhealthy” heartbeat and the extracted heartbeat data of the test subject. To support this determination, thresholds can be defined that establish tolerances around the simulated unhealthy heartbeat data to reduce the risk of false negatives. In other examples, step 264 can search the extracted heartbeat data for specific matching patterns to the simulated “unhealthy” heartbeat data that are known to be markers for adverse cardiac conditions or pathologies, as discussed above. Step 266 can then proceed in a fashion similar to that described above for step 256 of FIG. 25.

It should be understood that with the examples of FIGS. 25 and 26, the circuitry that performs any or all of steps 252-256 and/or any or all of steps 262-266 may include a processor that is programmed with software and/or firmware to carry out these operations. Moreover, such a processor can be located in close proximity to the sensor (e.g., on a printed circuit board or die that is housed in common, integrated packaging with the sensor) or it can be located remote from the sensor. For example, the sensor could include network connectivity, via wired and/or wireless networks, to a remote server system to communicate the sensor signal or extracted heartbeat data to that server system. The server system can include one or more processors that then perform steps 252-256 and/or steps 262-266. Iterative techniques are envisioned that would allow for machine learning and improved continuous tracking of a particular disease or patient.

Thus, unlike conventional techniques and technology, the novel modelling techniques and corresponding application to sensor technology disclosed herein is/are able to track cardiovascular health changes, thereby providing solutions for overcoming the lack of a standardized measurement device and protocol, and for the lack of uniform clinical interpretation.

Particularized sensor tuning is achieved by utilizing the closed-loop model in combination with sensor technology, thereby representing an improvement over the generalized sensors currently used in cardiovascular detection and/or monitoring applications. While such generalized sensors can detect forces, they are not capable of making a determination as to whether such forces are indicative of a particular cardiovascular function in the novel manner described herein. Because the closed-loop model is capable of being adapted to indicate presence of a particular cardiovascular disorder or tuned to the specific parameters of individualized patients, the closed-loop model, when combined with sensor technology, represents a vast improvement in the field.

In the present disclosure, all or part of the units or devices, or all or part of functional blocks in any block diagrams may be executed by one or more electronic circuitries including a semiconductor device, a semiconductor integrated circuit (IC) (e.g., such as a processor), or a large scale integration (LSI). The LSI or IC may be integrated into one chip, and may be constituted through combination of two or more chips. For example, the functional blocks other than a storage element may be integrated into one chip. The integrated circuitry that is called LSI or IC in the present disclosure is also called differently depending on the degree of integrations, and may be called a system LSI, VLSI (very large scale integration), or VLSI (ultra large scale integration). For an identical purpose, it is possible to use an FPGA (field programmable gate array) that is programmed after manufacture of the LSI, or a reconfigurable logic device that allows for reconfiguration of connections inside the LSI or setup of circuitry blocks inside the LSI. Furthermore, part or all of the functions or operations of units, devices or parts or all of devices can be executed by software processing. In this case, the software is recorded in a non-transitory computer-readable recording medium, such as one or more ROMs, RAMs, optical disks, hard disk drives, solid-state memory, and so on and so forth, having stored thereon executable instructions which can be executed to carry out the desired processing functions and/or circuit operations. For example, when the software is executed by a processor, the software causes the processor and/or a peripheral device to execute a specific function within the software. The system/method/device of the present disclosure may include (i) one or more non-transitory computer-readable recording mediums that store the software, (ii) one or more processors (e.g., for executing the software or for providing other functionality), and (iii) a necessary hardware device (e.g., an interface).

It should also be understood that when introducing elements of the present invention in the claims or in the above description of exemplary embodiments of the invention, the terms “comprising,” “including,” and “having” are intended to be open-ended and mean that there may be additional elements other than the listed elements. Additionally, the term “portion” should be construed as meaning some or all of the item or element that it qualifies. Moreover, use of identifiers such as first, second, and third should not be construed in a manner imposing any relative position or time sequence between limitations. Still further, the order in which the steps of any method claim that follows are presented should not be construed in a manner limiting the order in which such steps must be performed, unless such an order is inherent or explicit.

In view of the foregoing, it will be seen that the several advantages of the invention are achieved and attained. The embodiments were chosen and described in order to best explain the principles of the disclosure and their practical application to thereby enable others skilled in the art to best utilize the various embodiments and with various modifications as are suited to the particular use contemplated. As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. A system for sensing cardiovascular mechanisms for determination of a cardiovascular status, the system comprising:
a sensor configured to (1) sense cardiovascular forces of a subject and (2) generate a sensor signal representative of the sensed cardiovascular forces; and
a circuit configured to (1) extract cardiovascular function data from the sensor signal, (2) simulate cardiovascular function data based on a closed-loop cardiovascular model, (3) compare the extracted cardiovascular function data with the simulated cardiovascular function data, and (4) determine a cardiovascular status for the subject based on the comparison.

2. The system of claim 1 wherein the closed-loop cardiovascular model models cardiovascular function as a network of circuit components, and includes circuit components that model a heart, systemic circulation, pulmonary circulation and cerebral circulation.

3. The system of claim 2 wherein the circuit components include a plurality of resistors, capacitors, and inductors.

4. The system of claim 3 wherein the circuit components further include a plurality of voltage sources.

5. The system of claim 1 wherein the closed-loop cardiovascular model comprises a plurality of parameters, and wherein the circuit is programmable in response to user input to define the closed-loop cardiovascular model parameters.

6. The system of claim 3 wherein the circuit programmed in response to the user input defines the closed-loop cardiovascular model parameters with respect to physiological parameters of the subject.

7. The system of claim 1 wherein the circuit is configured to compare the extracted cardiovascular function data with the simulated cardiovascular function data to identify whether any deviations beyond a defined threshold exist between the extracted cardiovascular function data and the simulated cardiovascular function data.

8. The system of claim 1 wherein the circuit is configured to compare the extracted cardiovascular function data with the simulated cardiovascular function data to determine a degree of similarity between the extracted cardiovascular function data and the simulated cardiovascular function data.

9. The system of claim 1 wherein the circuit comprises a processor that executes the closed-loop cardiovascular model to simulate the cardiovascular function data.

10. The system of claim 1 wherein the extracted cardiovascular function data comprises extracted heartbeat data, and wherein the simulated cardiovascular function data comprises simulated heartbeat data.

11. The system of claim 1 wherein the cardiovascular status is the presence of a cardiovascular disease (CVD) of the subject.

12. The system of claim 1 wherein the cardiovascular status is the occurrence of a cardiovascular event of the subject.

13. The system of claim 1 wherein the sensor and the circuit are contained in an integral package housing.

14. The system of claim 1 wherein the circuit is further configured to generate (1) an extracted ballistocardiogram (BCG) from the extracted cardiovascular function data, and (2) a simulated BCG from the simulated cardiovascular function data.

15. The system of claim 1 wherein the sensor comprises an accelerometer.

16. A method for sensing cardiovascular mechanisms for determination of a cardiovascular status, the method comprising:
sensing cardiovascular forces of a subject;
generating a sensor signal representative of the sensed cardiovascular forces;
extracting cardiovascular function data from the sensor signal;
simulating cardiovascular function data based on a closed-loop cardiovascular model;
comparing the extracted cardiovascular function data with the simulated cardiovascular function data; and
determining a cardiovascular status for the subject based on the comparison.

17. The method of claim 16 wherein the closed-loop cardiovascular model models cardiovascular function as a network of circuit components, and includes circuit components that model a heart, systemic circulation, pulmonary circulation and cerebral circulation.

18. The method of claim 17 wherein the circuit components include a plurality of resistors, capacitors, and inductors.

19. An apparatus for modeling cardiovascular function, the apparatus comprising:
a processor configured to execute a closed loop cardiovascular model that simulates cardiovascular function as a network of circuit components with a plurality of tunable parameters.

20. The apparatus of claim 19 further comprising a memory configured to store data that represents the closed loop cardiovascular model, and wherein the network of circuit components includes circuit components that model a heart, systemic circulation, pulmonary circulation and cerebral circulation.
